(19) United States
(12) Patent Application Publication
(10) Pub. No.: US 2012/0258474 A1
(43) Pub. Date: Oct. 11, 2012

(54) METHODS OF DETECTING DNA DAMAGE

(75) Inventors: Peter James Tatnell, Cardiff (GB); Jeffrey Kenneth Horton, Cardiff (GB); Nicholas Thomas, Cardiff (GB)

(73) Assignee: GE HEALTHCARE UK LIMITED, LITTLE CHALFONT (GB)

(21) Appl. No.: 13/515,021

(22) PCT Filed: Dec. 8, 2010
(86) PCT No.: PCT/EP2010/069157

§ 371 Date: Jun. 11, 2012

(30) Foreign Application Priority Data
Dec. 11, 2009 (GB) 0921712.6

(51) Int. Cl. G01N 33/53; G01N 33/573; C12Q 1/42; G01N 21/55; C12Q 1/46

(52) U.S. Cl. 435/7.21; 435/7.1; 435/29; 435/20; 435/21; 435/7.4

(57) ABSTRACT

The present invention relates to methods of detecting agents that cause or may potentiate DNA damage, and to assays that may be employed in such methods. In particular, the invention relates to methods of detecting DNA damage in in vitro cultures of human cells.

### FIELD OF THE INVENTION

The present invention relates to methods of detecting agents that cause or may potentiate DNA damage, and to assays that may be employed in such methods. In particular, the invention relates to in vitro method of measuring the genotoxicity of an agent in human cells.

### BACKGROUND TO THE INVENTION

The identification of human genotoxic hazard is an important step in the development of pharmaceuticals. To this end, genotoxicity testing is performed to ensure safety during clinical trials and during the treatment of general patient populations (Cimino, Environ. Mol. Mutagen, 2006, 47, 362-390).

The current core test battery for hazard identification, agreed upon by regulatory agencies and pharmaceutical companies from the United States, the European Union, and Japan, consists of a series of in vitro and in vivo genotoxicity assays (ICH, S2B, 1997, International Conference on Harmonisation of Technical Requirements for Registration of Pharmaceuticals for Human Use (ICH), available at ich.org; Cimino, Environ. Mol. Mutagen, 2006, 47, 362-390). Furthermore the European Union has enacted (in 2007) a new chemical regulation called REACH (Registration, Evaluation and Authorization of Chemicals), which promises to be the most complex and comprehensive regulatory effort covering chemicals, ever instituted. Within this law, the requirements for toxicology are very important, because they result in the highest requirement for funding and the need for experimental animals. In addition, reproductive and developmental considerations may result in the restriction of many substances that are now in widespread use. Although REACH has what appears to be stringent requirements for experimental animal studies, the law discourages the use of vertebrate animals in testing, requiring laboratories to consider alternative methods. Many of the established alternative animal methods are often problematic and thus there is a great need for better and improved laboratory methods that can accurately predict the result of a chemical insult. Thus, there exists a great need for the introduction and validation of new cell-based methods to assess the potential toxic effect of chemicals, pharmaceuticals, cosmetics and foodstuffs on humans. Ideally, these methods should involve in vitro, laboratory testing of potential genotoxic agents.

DNA Damage and Mutation

DNA damage is induced by a variety of agents such as ultraviolet light, X rays, free radicals, methylating agents and other mutagenic compounds. DNA damage can also be caused indirectly either by agents that affect enzymes and proteins which interact with DNA (including polymerases and topoisomerases) or by pro-mutagens (agents that can be metabolized to become mutagenic). Any of these agents may cause damage to the DNA that comprises the genetic code of an organism and cause mutations in genes. In animals, such mutations can lead to carcinogenesis or may damage the gametes to give rise to congenital defects in offspring. Such DNA damaging agents are collectively known as genotoxins.

These DNA damaging agents may chemically modify the nucleotides that comprise DNA and may also break the phosphodiester bonds that link the nucleotides or disrupt association between bases (T-A or C-G). To counter the effect of these DNA damaging agents, cells have evolved a number of mechanisms. For example, the SOS response in E. coli is a well-characterized cellular response induced by DNA damage in which a series of proteins are expressed, including DNA repair enzymes. In mammals, nucleotide excision and base excision repair mechanisms play a prominent role in maintaining the integrity of DNA, and are the primary mechanism for removal of bulky DNA adducts and modified bases.

There are numerous circumstances when it is important to identify what agents may cause or potentiate DNA damage. It is particularly important to detect agents that cause DNA damage when assessing whether it is safe to expose a person to these agents. For instance, a method of detecting these agents may be used as a genotoxicity assay for screening compounds that are candidate medicines, food additives or cosmetics to assess whether or not the compound of interest induces DNA damage. Alternatively, methods of detecting DNA damaging agents may be used to monitor contamination of water supplies with pollutants that contain mutagenic compounds.

PigA Gene

The PigA gene is located on the X-chromosome. This is a highly desirable feature, since one functional copy means that only a single mutational event is necessary to produce a phenotype, which can be readily detected. More specifically, the PigA gene product is essential for the biosynthesis of phosphatidylinositol glycan (GPI) anchors. Mutations can give rise to either a non-functional or a PigA gene product possessing a reduced enzymatic activity and result in the absence or reduced membrane expression of GPI-linked proteins.

In peripheral blood cells the absence of GPI-anchored proteins lead to paroxysmal nocturnal hemoglobinuria (PNH). PNH is an acquired genetic disorder that affects 1 to 10 million individuals worldwide. The molecular basis of PNH is a somatic PigA gene mutation within a bone marrow stem cell. PNH usually affects erythrocytes, granulocytes and monocytes. In a minority of cases, the lymphocyte lineage is also affected, and only a few case reports have documented lymphocytes to be the cell lineage affected. Several GPI-linked proteins, especially CD55 and CD59 have been studies intensely. In fact, flow cytometry-based techniques, which measure the frequency of CD55 and/or CD59 deficient red blood cells, are replacing the Ham's Acid Hemolysis (Ham's) test for PNH diagnosis.

The PigA (or phosphatidylinositol glycan anchor biosynthesis, class A) gene encodes a protein required for synthesis of N-acetylglucosaminyl phosphatidylinositol (GlcNAc-PI), the first intermediate in the biosynthetic pathway of GPI anchor. The GPI anchor is a glycolipid found on many cells and which serves to anchor proteins to the cell surface.

The Pig Locus and Paroxysmal Nocturnal Hemoglobinuria

The PigA gene locus possesses a high degree of inter-species conservation and somatic mutations within the locus are linked to Paroxysmal Nocturnal Hemoglobinuria (PNH, Norris et al, 1994, Blood, 83, 816-821 and Nishimura et al, 1999, Am. J. Hematol, 62, 175-182). In this disease, decay accelerating factor (DAF or CD55) and CD59 are not anchored to red blood cells because of the faulty GPI linkage. In the absence of these GPI-linked proteins on the cell surface, the complement system will cause cellular lysis and high numbers of red blood cells are destroyed leading to hemoglobinuria.

Many cell surface proteins are anchored to the membrane by a GPI-anchor via a covalent attachment to the C-terminus of the peptide. The core of the GPI anchor consists of multiple components and its biosynthesis involves multiple genes. Some of these genes are represented by different complementation classes of GPI anchor-deficient mutant cells derived from human and rodent cell lines (Stevens and Raetz,. J. Biol. Chem. 1991, 266: 10039-10042; Sugiyama, et al., J. Biol. Chem. 1991, 266, 12119-12122; Hirose, et al., Proc. Nat. Acad. Sci. 1992, 89, 6025-6029).

By expression cloning methods, Miyata, et al., 1993, (Science, 259, 1318-1320) cloned a gene they termed PigA (for phosphatidylinositol glycan class A) and found that the gene encodes a predicted protein of 484 amino acids containing a single predicted transmembrane-spanning domain.

Watanabe, et al., 1996 (J. Biol. Chem., 271, 26868-26875) demonstrated that the PigA and PigH proteins form a protein complex and are subunits of the GPI GlcNAc transferase of the endoplasmic reticulum (ER). They showed that PigA is an ER transmembrane protein with a small luminal domain and a large cytoplasmic domain. The luminal domain targets the protein to the rough ER, while the cytoplasmic domain has homology to the bacterial GlcNAc transferase RfaK. The authors concluded that the first step of GPI anchor synthesis occurs on the cytoplasmic side of the ER membrane.

Using immuno-precipitation experiments, Watanabe et al. 1998 (EMBO J., 17, 877-885) demonstrated that PigQ associates specifically with PigA, PigC, and PigH and that all 4 proteins form a complex that has GPI-GlcNAc transferase (GPI-GnT) activity in vitro.

Paroxysmal nocturnal hemoglobinuria (PNH) is an acquired hematologic disorder characterized by complement-mediated haemolysis and cloned expansion of affected cells of various haematopoietic lineages that are thought to be derived from an abnormal multi-potential haematopoietic stem cell. Although not an inherited genetic disease, PNH is an acquired genetic disorder. The affected clone endows all its descendants, red cells, leukocytes (including lymphocytes), and platelets, with the mutated gene. These mutant cells grow “side-by-side” with normal cells, creating a hematologic mosaic in which the proportion of abnormal erythrocytes in the blood determines the severity of the disease. Its clinical hallmark, black urine on arising from sleep, is testimony to intravascular haemolysis during the night.

Haemolysis also occurs after blood from a patient with PNH is mixed with acidified serum or ordinary table sugar; this is the basis of the Ham and sugar-water tests for PNH. Biosynthesis of the GPI anchor is deficient in the affected cells from patients with PNH (Mahoney et al., Blood 1992, 79: 1400-1403; Hirose et al., Proc. Nat. Acad. Sci. 1992, 89, 6025-6029) leading to deficient surface expression of multiple GPI-anchored proteins, such as decay-accelerated factor (CD55) and CD59 both of which play roles in the protection of red cells from the action of complement. Venous thrombosis, an increased incidence of leukemia arising from the affected cells, and a tendency for association with aplastic anemia are other features of the disease.

Socie et al. 1996 (Lancet, 348, 573-577) reported a case-control study on the 7 factors that they found to be significantly associated with survival in PNH patients (6 negative and 1 positive). Risk factors affecting 220 patients in the French population (diagnosed by a positive Ham test) were used in this multivariate analysis. The 6 factors associated with decreased survival were the development of thrombosis, progression to pancytopenia, myelodysplastic syndrome or acute leukemia, ages over 55 years at diagnosis, multiple attempts at treatment, and thrombocytopenia at diagnosis. The only protective factor found was, surprisingly, a history of aplastic anemia antedating the diagnosis of PNH. The mean survival was found to be 15 years (Rosse 1996 Lancet, 348, 560).

Ueda et al. 1992 (Int. Immun. 4, 1263-1271) established affected B lymphocyte cell lines from 2 patients with PNH, and Takahashi et al. 1993 (J. Exp. Med. 177, 517-521) demonstrated that the early step of GPI anchor biosynthesis was deficient in these cells. Complementation analysis by somatic cell hybridization with GPI-deficient mutant cell lines showed that these PNH cell lines belong to complementation class A, which is known not to synthesize GlcNAc-PI. Takeda et al. 1993 (Cell, 73, 703-711) found that transfection of PigA cDNA into affected B lymphoblastoid cell lines restored their surface expression of GPI-anchored proteins. Further analysis demonstrated that the PigA transcript was missing or present in very small amount in cell lines established from 1 patient, but that in a cell line established from another patient, deletion of thymine in a 5-prime splice site was associated with deletion of a PigA exon located immediately 5-prime to the abnormal splice donor site.

By fluorescence in situ hybridization with genomic PigA probes on R-banded chromosomes, Takeda et al. 1993 (Cell, 73, 703-711) demonstrated that the PigA gene resides on Xp22.1. Since 1 of the patients studied was female, they concluded that the mutant gene must reside on the active X chromosome. Affected cell lines established from 5 other patients with PNH were shown to belong to complementation group class A, indicating that the target gene is the same in most, if not all, patients with PNH. This can account for the behaviour of the deficiency as a dominant in hemizygous males and in females with the mutant gene on the active X chromosome in a given lymphoblastoid cell line.

It has been shown that all cases of PNH appear to have a defect in the PigA gene, but the causative mutation has in all instances been unique. That many different mutations of PigA may result in PNH may not be surprising since they arise as somatic mutations. It has been suggested that a germ-line mutation resulting in defects in this biosynthetic pathway would be lethal. lida et al. 1994 (Blood, 83, 3126-3131) reported that the PigA gene is at least 17 kb long and possesses 6 exons. They sequenced the exon-intron boundaries and described the characteristics of the 5-prime promoter region.

Ware et al. 1994 (Blood, 83, 3753-3757) used an inter-specific cross to demonstrate that the PigA gene in the mouse is also located on the X chromosome. Kawagoe et al. 1994 (Genomics, 23, 566-574) also mapped the mouse gene to the X chromosome in a region that shows homology of synteny to the Xp22.1 in the human genome. The deduced amino acid sequence of the mouse protein is 88% identical with that of the human protein. Transfection of the mouse cDNA complemented the defects of both a PigA-deficient murine cell line and a PigA-deficient human cell line, demonstrating that functions of the mouse and human proteins are conserved. Like the human gene, the mouse gene has 6 exons and spans approximately 16 kb. Database analysis demonstrated that a yeast gene, Spt14, is homologous to PigA.

Bessler et al. 1994 (Lancet, 343: 951-953) reported an elegant series of experiments in two patients with PNH, each of whom had two independently arising PNH clonal lines. All four clones had an entirely separate mutational basis. Bessler et al. 1994 (Lancet, 343: 951-953) presented these observations as further support for positive selection of PNH clones with inhibition of normal haematopoiesis. With regard to the known association between PNH and aplastic anemia, their suggestion was that aplastic anemia inhibits normal haematopoiesis but that PNH cell clones are unaffected by this inhibition. Haematopoiesis, albeit of an abnormal clone, continues; an example of gene therapy in the wild.

In granulocytes from three of fifteen patients with PNH, Miyata et al. 1994 (New Eng. J. Med. 330, 249-255) demonstrated size abnormalities of PigA transcripts with different patterns, and in one patient a very low expression level of the PigA transcript was observed. Although eleven patients had transcripts of normal size, transfection assay demonstrated that in each patient some of the transcripts were non-functional. The percentage of non-functional PigA transcripts correlated with the percentage of affected granulocytes (P<0.001). Sequence analysis demonstrated somatic mutations in two of the patients: deletion of a T and insertion of an A. The PigA gene as the site of the defect in all patients with PNH is remarkable in light of the fact that PigA is but one of at least ten genes involved in GPI synthesis.

The location of the gene on the X chromosome is probably responsible: somatic mutation in only one X chromosome is necessary to produce the mutation in a male cell or for that matter in a female cell if it occurs on the active X chromosome. Bessler et al. 1994 (EMBO J., 13: 110-117) reviewed the evidence that PNH is caused by somatic mutations in the PigA gene. They demonstrated a somatic point mutation in 4 cases which, with the 2 mutations reported by Takeda et al. 1993 (Cell 73, 703-711), brought to six the number in which formal proof of the absence of normal PigA gene product has been shown to produce the PNH phenotype.

Savoia et al. 1996 (Hum. Genet., 97, 45-48) found a novel mutation in the PigA gene in each of 3 Italian patients. In each case, the mutation caused premature termination in the translation of the PigA protein. Nafa et al. 1995 (Blood, 86, 4650-4655) identified 15 different somatic mutations in 12 patients with PNH; 10 of them caused frame-shifts. In each of 3 patients, 2 independent mutations were identified. Whereas G6PD mutations are virtually all single base pair changes that result in single amino acid replacements, most PigA mutations are insertion-deletion mutations that generally cause such frame-shift mutations. The authors stated that the predominance of null mutations probably reflects the fact that the total absence of GPI-linked proteins provides a relative survival or growth advantage to the affected cells that is greater than that when the deficiency of GPI-linked proteins is only partial.

Nafa et al. 1998 (Blood Cells Molec. Dis. 24, 370-384) described 28 previously unreported mutations. They confirmed that somatic mutations are spread throughout the entire coding region of the PigA gene and that most frame-shift mutations produce a non-functional PigA protein. In addition, they found one total deletion of the PigA gene, and two short nucleotide duplications. Although mutations are spread throughout the entire coding region, they observed more mis-sense mutations in exon 2 than in other exons.

Treatment of severe aplastic anemia with anti-thymocyte globulin (ATG) and cyclosporin leads to clinical remission in a large proportion of patients. However, as many as 10 to 57% of these patients develop PNH. The secondary PNH tends to be more indolent than classic PNH. Nagarajan et al. 1995 (Blood, 86, 4656-4661) studied 4 patients with this form of secondary PNH. All four of their aplastic patients who developed PNH had a negative Ham test at diagnosis of aplastic anemia. A positive Ham test developed within three months after ATG/cyclosporine administration in two of the four; after immunosuppressive therapy, one developed a positive test at six months and another at eighteen months. All four patients remained transfusion-independent with no thrombotic episodes after mean follow-up of thirty months. A mutation in the PigA gene was identified in each of the four patients. Nagarajan et al. 1995 (Blood, 86, 4656-4661) concluded that the seeming indolent nature of secondary PNH merely reflects early detection.

On the basis of a group of eighty consecutive patients with PNH who were referred to Hammersmith Hospital, London, between 1940 and 1970, Hillmen et al. 1995 (New Eng. J. Med. 333: 1253-1258) defined the natural history of this disorder. The median age of patients at the time of diagnosis was forty two years (range, 16 to 75), and the median survival after diagnosis was ten years, with twenty two patients (28%) surviving for 25 years. Sixty patients had died; twenty eight of the forty eight patients for whom the cause of death was known, died from either venous thrombosis or hemorrhage. Thirty-one patients (39%) had one or more episodes of venous thrombosis during their illness. Of the thirty five patients who survived for 10 years or more, twelve had a spontaneous clinical recovery. No PNH-affected cells were found among the erythrocytes or neutrophils of the patients in prolonged remission, but a few PNH-affected lymphocytes were detectable in three of the four patients tested. Leukemia did not develop in any of the patients. The patients had been treated with supportive measures, such as oral anticoagulant therapy after established thromboses and transfusions. Hillmen et al. 1995 (New Eng. J. Med. 333: 1253-1258) stated that the occurrence of spontaneous long-term remission must be taken into account when considering potentially dangerous treatments, such as bone marrow transplantation (BMT). Platelet transfusion should be given, as appropriate, and long-term anticoagulation therapy should be considered for all patients.

Luzzatto and Bessler 1996 (Curr. Opin. Hemet., 3, 101-110) and Luzzatto et al. 1997 (Cell 88, 1-4) reviewed the topic of PNH and gave a survey of the more than 100 somatic mutations in the PigA gene that had been identified in patients with this disorder. Luzzatto et al. 1997 (Cell 88, 1-4) concluded that two different causes are required to give the clinical phenotype of PNH: i) that we now understand, namely a somatic mutation in the PigA gene; and ii) that can only be defined as a specific type of bone marrow failure. The implications of this testable model are that i) alone would produce PNH clones of no clinical significance, which may be lurking in normal people, whereas ii) alone would give the clinical picture of aplastic anemia. It is only when i) and ii) coexist in the same person that we see a clinical phenotype of PNH.

PNH is hypothesized to have a conditional growth or survival advantage and environment that is injurious to hematopoietic cells through a GPI-mediated mechanism (Rotoli and Luzzatto, Clin. Haemat. 1989, 2, 113-138). For instance, if the damage was caused by auto-reactive T cells or by natural killer cells, as has been suggested to be the case in aplastic anemia, one could speculate that this happens by virtue of these cells triggering an apoptotic pathway by interacting with a GPI-linked molecule normally present on the surface of hematopoietic stem cells. Under this hypothesis, it is obvious that PNH cells, being invulnerable to this special kind of injury, would be at an advantage as long as the offending T cells or natural killer cells are present; whereas they would revert to being neutral or even at a disadvantage once such offending cells are no longer present.

In the title of a review on PNH, Nishimura et al. 1999 (Am. J. Hemat. 62, 175-182) referred to the paradox of referring to the disorder as an ‘acquired genetic disease.’ Although many of the clinical manifestations (for example, hemolytic anemia) of PNH can be explained by a deficiency of GPI-anchored complement regulatory proteins such as CD55 and CD59, it was unclear why PNH clonal cells dominate hematopoiesis and why they are prone to evolve into acute leukemia.

Brodsky et al. 1997 (Proc. Nat. Acad. Sci. 94, 8756-8760) found that PigA mutations confer survival advantage by making cells relatively resistant to apoptotic death. When placed in serum-free medium, granulocytes and affected CD34⁺ cells from PNH patients survive longer than their normal counterparts. PNH cells were also relatively resistant to apoptosis induced by ionizing irradiation. Replacement of the normal PigA gene in PNH cell lines reversed the cellular resistance to apoptosis.

Brodsky et al. 1997 (Proc. Nat. Acad. Sci. 94, 8756-8760) speculated that apoptosis inhibition may be the principal mechanism by which PNH cells maintain a growth advantage over normal progenitors and could play a role in the propensity of this disease to transform into more aggressive hematologic disorders. The work also suggested that GPI anchors are important in regulating apoptosis.

The clinical association between PNH and acquired aplastic anemia (AAA), and the observation that, as in AAA, PNH patients have decreased hematopoietic progenitors, may be taken to suggest a common patho-genetic process. There is strong evidence that AAA is an autoimmune disease and, as for AAA, bone marrow failure in PNH can be treated successfully with immuno-suppression; thus, autoimmunity is likely to play a role in PNH as well. Specifically, it has been hypothesized that an autoimmune attack on normal stem cells targets a GPI-linked molecule and therefore preferentially spares the PNH stem cell, which thus has a growth or survival advantage (or both) in this abnormal environment.

Using flow cytometric analysis of granulocytes, Araten et al. 1999 (Proc. Nat. Acad. Sci. 96, 5209-5214) identified cells that had the PNH phenotype (lack of expression of proteins linked to the membrane by a GPI anchor) at an average frequency of 22 per million in 9 normal individuals. These rare cells were collected by flow sorting, and exons 2 and 6 of the PigA gene were amplified by nested PCR. The authors identified PigA mutations in 6 cases. PNH red blood cells were also identified at a frequency of 8 per million. Thus, a small numbers of clones existed commonly in normal individuals that possess PigA mutations, showing clearly that PigA gene mutations are not sufficient for the development of PNH.

Because PigA encodes an enzyme essential for the expression of a host of surface proteins, the PigA gene provides a highly sensitive system for the study of somatic mutations in hematopoietic cells. Araten et al. 1999 (Proc. Nat. Acad. Sci. 96, 5209-5214) reported the finding of a try98ser mutation in a 61-year-old man being phlebotomized for hemochromatosis. This was confirmed in samples taken 8 weeks apart. This same mutation had been reported in a patient with PNH (Savoia et al., 1996, Hum. Genet. 97, 45-48). Thus, the very same PigA mutation that caused PNH in one person did not cause PNH in another person.

Conditions favouring mutation in cases of PNH have been suggested by the coexistence of multiple clones with different mutations in the PigA gene and the appearance of leukemic clones in patients. Horikawa et al. 2002 (Blood, 99, 24-29) tested this hypothesis by examining the frequency of mutations in the HPRT gene and identified by both resistance to 6-thioguanine and gene analysis. T-cell colonies resistant to 6-thioguanine formed in methylcellulose culture were found in eight (67%) of twelve PNH patients and three (18%) of seventeen age-matched healthy volunteers. Incidence of resistant colonies ranged from forty to three hundred and sixty seven [mean 149] in the eight patients and from one to sixteen [mean 7] in the three healthy donors. Unlike PNH cells, 6-thioguanine-resistant cells express CD59, indicating that the HPRT mutation did not occur in PNH clones. No correlation was noted between HPRT mutation frequency and content of therapy received by the patients. The authors concluded that in PNH patients, conditions exist that favour the occurrence of diverse somatic mutations in blood cells.

To determine whether PNH blood cells are also present in patients with inherited aplastic anemia, Keller et al. 2002 (Brit. J. Haemat. 119, 830-832) screened a large group of patients with Shwachman-Diamond syndrome. None of the patients analyzed had detectable circulating PNH blood cells, indicating that bone marrow failure in Shwachman-Diamond syndrome does not select for PNH progenitor cells.

In patients with PNH, Hillmen et al. 2004 (New Eng. J. Med. 350, 552-559) tested the clinical efficacy of eculizumab, a humanized antibody that inhibits the activation of terminal complement components. They found that the drug was safe and well tolerated by the patients. This antibody against terminal complement protein C5 reduced intravascular hemolysis, hemoglobinuria, and the need for transfusion, with an associated improvement in the quality of life.

PNH is extremely rare in children. Van den Heuvel-Eibrink et al. 2005 (Brit. J. Haemat. 128, 571-577) reported 11 Dutch pediatric PNH patients with a median age of 12 years. In 7 cases, PNH was associated with aplastic anemia and in 4 with myelodysplastic syndrome. Information on the molecular defect was not provided.

Hu et al. 2005 (Blood, 105, 3848-3854) confirmed the finding that mutations of the PigA gene are relatively common in normal hematopoiesis; however, they demonstrated that these mutations occur in differentiated progenitor cells rather than in hematopoietic stem cells.

Pseudogenes

In the course of analyses of PigA genetic alterations in PNH patients, Yu et al. 1994 (Brazilian J. Med. Biol. Res. 27, 195-201) amplified PigA transcripts expressed in affected lymphocytes by RT-PCR and unexpectedly found a product differing from the authentic PigA mRNA by 126 nucleotide exchanges and five deletions in the coding region. This pseduogene was termed PigAP. Nagarajan et al. 1995 (Hum. Genet. 95, 691-697) showed that mRNA with this sequence was co-expressed with PigA mRNA in a wide range of cell types. Mapping of genomic DNA from human/rodent hybrids showed that this sequence was derived from an intron-less pseudogene that was encoded by chromosome 12.

Expressed duplicated genes had been described for a number of X-linked genes. In the case of pyruvate dehydrogenase a duplicated gene is located on chromosome 4. This gene is transcribed and fully functional, encoding a testis-specific form of the enzyme. The adenine nucleotide translocase genes located on Xp, are also duplicated on chromosome 9. The functional status of this gene is unknown. The X-linked gene for phosphoglycerate kinase has a processed gene located in close proximity and two related autosomal genes are found on chromosomes 19 and 6. The nearby X-linked gene is transcribed but inactive.

The identification of a stop codon at position 243 in the mRNA sequence of the PigAP gene on chromosome 12 indicates that if the mRNA is transcripted, and survives regulatory mechanism, the protein product is probably not functional.

GPI-Linked Proteins

Glycosylphosphatidylinositol-linked proteins are known to play an important role in biological membranes (Low and Saltiel, 1988 Science, 239, 268-275). These molecules can serve as the sole means by which many cell-surface proteins are anchored to the membrane. This mechanism involves a covalent linkage from the protein to an oligosaccharide which is in turn glycosidically linked to phosphatidylinositol. The resulting class of membrane glycophospholipids, termed glycosyl-phosphatidylinositols, have now been detected in a wide variety of eukaryotic cells (see Low et al 1986, Trends Biochem, Sci, 11, 212-215, and, Cross, 1987 Cell, 48, 179-181).

A specific role for phosphatidylinositol in the attachment of proteins to membranes was first demonstrated for alkaline phosphatase by Low & Zilversmit, 1980, (Biochemistry, 19, 3913-3918). Indeed, it has been shown that treatment of membranes with highly specific bacterial phospholipases, the phosphatidylinositol-specific phospholipases C (PI-PLC), released a number of hydrolytic enzymes (for example, alkaline phosphatase, 5′-nucleotidase, and acetylcholinesterase) from membranes in a water-soluble, non-aggregated form that retained full activity but was unable to re-associate with the membrane (Futerman et al, 1985, Biochem. J., 226, 369-377). The release of these proteins was not a consequence of non-specific alterations in the membrane microenvironment since PI-PLC could also remove the hydrophobic attachment site from proteins solubilized from the membrane by detergents or organic solvents.

Thus, the liberation of these proteins from the membrane was the result of a selective removal of the membrane anchoring domain by PI-PLC, leading to the proposal that membrane attachment was entirely the result of a covalently linked phosphatidylinositol molecule (Low & Zilversmit, Biochemistry, 1980, 19, 3913-3918, Futerman et al, Biochem. J. 1985, 226, 369-377). This concept was supported by the detection of covalently attached myo-inositol in acetylcholinesterase (AChE) from the electric organ of the electric ray (Futerman et al, 1985, Biochem. Biophys. Res. Commun.,129, 312-317) and alkaline phosphatase from human placenta (Low et al, 1987, Biochem. J., 241, 615-619).

The ability of bacterial PI-PLC to release proteins attached to the membrane by this mechanism has permitted the identification of a number of phosphatidylinositol-anchored proteins (see Table 1). This group of proteins is both evolutionarily and functionally diverse. It includes seven distinct hydrolytic enzymes, a complement regulatory protein [decay accelerating factor (DAF)], neural and lymphocyte cell adhesion molecules (N-CAM and LFA-3), a protective coat protein in the parasitic protozoan Trypanosoma brucei [variant surface glycoprotein (VSG)], and the scrapie prion protein (PrP), as well as a number of antigens. In the specific case of myelin basic protein, the phosphoinositide may be attached to only a small proportion of the protein molecules (Smith et al, 1987, Biochem. J. 248, 285-288).

Glycosylphosphatidylinositol (GPI anchor) is a glycolipid that can be attached to the C-terminus of a protein during post-translational modification. It is composed of a phosphatidylinositol group which can be linked through a carbohydrate containing linker (glucosamine and mannose glycosidically bound to the inositol residue) to the C-terminal amino acid of a mature protein. The two fatty acids within the hydrophobic phosphatidyl-inositol group anchor the protein to the cell membrane.

Glypiated (GPI-linked) proteins contain a signal peptide, thus directing them into the ER. The C-terminus is composed of hydrophobic amino acids which stay inserted in the ER membrane. The hydrophobic end is then cleaved off and replaced by the GPI-anchor. As the protein processes through the secretory pathway, it is transferred via vesicles to the Golgi apparatus and finally to the extracellular space where it remains attached to the exterior leaflet of the cell membrane (FIG. 1). Since the glypiation is the sole means of attachment of such proteins to the membrane, cleavage of the group by phospholipases will result in controlled release of the protein from the membrane. The latter mechanism is used in vitro, i.e. the membrane proteins released from the membranes in the enzymatic assay are glypiated protein.

Phospholipase C (PLC) is an enzyme that is known to cleave the phospho-glycerol bond found in GPI-anchored proteins. Treatment with PLC will cause release of GPI-linked proteins from the outer cell membrane. The T-cell marker Thy-1, acetylcholinesterase, as well as both intestinal and placental alkaline phosphatase are known to be GPI-linked and are released by treatment with PLC. GPI-linked proteins are thought to be preferentially located in lipid rafts, suggesting a high level of organization within plasma membrane microdomains.

Defects in the synthesis of GPI anchors occur in the disease, PNH. In this disease decay accelerating factor (DAF or CD55) and CD59 are not properly anchored to red blood cells because of the faulty GPI linkage. Without the presence of these cell surface proteins, the complement system can lyse the cell and high numbers of RBC's are destroyed leading to hemoglobinuria. Other cells are not affected by complement attack because they possess transmembrane forms of DAF and CD59.

A number of identified phosphatidylinositol-anchored proteins and thus targets for genotoxic agents involving the PigA gene linked to GPI-linked targeted proteins with their associated cellular functions are outlined in Table 1.

TABLE 1
Targets for genotoxic agents involving the PIGA gene and
GPI-linked targtted proteins

| Molecule | Function |
|---|---|
| CD14 | LPS receptor |
| CD16 | Low affinity receptor for IgG |
| CD24 | Signal transducing molecule |
| CD48 | BLAST-1 adhesion molecule, counter rceptor |
| | for CD2 |
| CDw52 | Surface antigen on T and B cells |
| CD55 | Decay Accelerating Factor |
| CD56 | NCAM-120 |
| CD58 | LFA3 adhesion molecule |
| CD59 (HRF) | Inhibitor of complement attack complex formation |
| CD66acd&e | CEACAM family granulocyte specific |
| CD67 | Immunoglobulin, CEA superfamily member |
| CD73 | Ecto-5′-nucleotidase |
| CD87 | uPA receptor |
| Thy-1 CD90 | T cell antigen |
| CD108 | Semaphorin 7a JMH blood group antigen |
| CD157 | Bone marrow stromal cell antigen 1 |
| AChE | Acetylcholinesterase |
| NAP | Alkaline phosphatase |
| uPAR | Urokinase-type plasminogen (monocyte |
| | activation antigen) |
| JMH protein | Blood group antigen |
| GDNFR | Glial cell line-derived neurotrophic factor receptor |
| CNTFR | Ciliary neurotrophic factor receptor |
| TAG-1 | Transient axonal glycoprotein-1 |
| Prion protein | Expressed in the brain and several other tissues |
| Glypican family | GPI-anchored proteoglycans, found in |
| | developing embryo, tubular epithelial cells in the |
| | kidney and proliferating neuroepithelial cells |
| | in the brain |
| Semaphorin 7 | Brain and spinal cord, neuronal guidance |
| CEA | Carcinoembryonic antigen |
| GFR | Neuronal survival |
| Ly-6 | Involved in lymphocyte activation |
| Transferrin receptor | Iron transport in the epithelium |
| Contactin (F3) | Adhesion molecule |
| T-cadherin | Adhesion molecule in vascular smooth muscle cells |

In mammals, greater than 200 different cell surface proteins are anchored to the membrane via attachment to a GPI moiety. These proteins include enzymes and receptors and represent targets for the mutation assay described herein. Furthermore GPI-linked surface proteins can be transferred or inserted into membranes readily. Thus it is possible to insert any protein into the cell surface via a GPI-anchor using recombinant DNA or gentic engineering techniques. (Medof et al, 1996, FASEB J., 10, 574-586).

The present invention identifies procedures that can be employed as simple in vivo or in vitro gene mutation assays that can be used to evaluate agents such as electromagnetic radiation and/or chemical compounds (e.g. drug, chemical, cosmetic or physical agents) for genetic toxicity. In addition, the methods described here are fast, reliable and accurate and can be carried out without the the need for transgenic cell or animal models.

The presence of GPI-linked molecules can be measured either on the cell surface, in cellular extracts, or, in a soluble form (after PLC treatment) in cell culture supernatants using specific antibodies-tagged with reporters. Such antibody mediated binding events can also be monitoring using surface plasmon resonance (SPR, Biacore, GE Healthcare). An alternative approach, involves the direct measurement of specific enzymatically active GPI-anchored proteins using luminescent substrates.

In addition, quantitative RT-PCR could also be used to monitor mutations within the PigA gene promoter region that cause changes in the mRNA expression levels generated in response to the exposure of cells to genotoxic compounds.

A quantitative immunocytochemistry multiplexed HCA approach represents an additional method where the presence of GPI-proteins may be correlated with other cytotoxic markers such as, viability, micro-nuclei analysis, mitochondrial membrane integrity. With standard cellular vital dyes, the use of antibodies tagged with fluorescent reporter dyes such as Cy5 or Cy3 (GE Healthcare) facilitates the formation of a multiplexed HCA cellular image analysis.

Supplementary and alternative approaches, involve the direct measurement of endogenous enzymatically active GPI-anchored proteins such as: acetylcholinesterase and alkaline phosphatase. This is achieved using luminescent substrates (for example, acetylcholine/ATP and dioxetane-based substrates respectively), whereby measurements are made on a suitable plate reader or imager involving CCD cameras (for example LEADseeker, GE Healthcare, ViewLux Perkin Elmer). The Vector Red substrate for alkaline phosphatase can also be used for cell or tissue staining for use in high-content analysis optical cell imaging.

Cell Imaging

Using a high-content cellular imager (for example InCell 2000, GE Healthcare) in multiplexing mode, it is possible to detect a number of different fluors tethered to either different specific antibodies or to compartment localisation cellular vital dyes. These fluors would be used to detect and measure several target molecules simultaneously. Thus the instrument has the potential of measuring, using quantitative immunocytochemistry, the effect of a toxic insult on several different markers from the same sample, thus enabling a toxicity profile of an agent to be constructed. Furthermore, the approach described below has an additional embodiment, in that using this multiplex approach; the method has the capability of discriminating between several different selective biomarkers.

As the cultured cells (transformed lines or primary) respond to a toxic insult, these reporters can simultaneously detect and measure several cell/tissue specific molecules present in the specific cell type, resulting in the availability of significant amount of information to the investigator. In addition the ability to generate quantitative data (whether derived from immunocytochemistry or gene reporter assays) may allow the determination of sub-lethal dosage levels. This may be of significant value for the drug development, agrochemical or cosmetic industries.

Prior Art

Various methods for assessing a compounds genotoxcity, such as the Ames Test, the in vitro micronucleus test and the mouse lymphoma assay (MLA), for determining the toxicity of an agent are known but are unsatisfactory for a number of reasons. For instance, incubation of samples can take many weeks, when it is often desirable to obtain genotoxic data in a shorter time-frame. Furthermore, many known methods of detecting DNA damage (including the Ames Test and related methods) monitor DNA damage, as an endpoint, either in the form of mis-repaired DNA (mutations and recombinations) or un-repaired damage in the form of fragmented DNA. However, most DNA damage is repaired before such an end-point can be measured and lasting DNA damage only occurs if the conditions are so severe that the repair mechanisms have been saturated.

US20070042343 (Merck & Co) describes a forward mutation assay based on 5-fluorouracil (5-FU) resistance, which utilizes a strain of Salmonella typhimurium derived from the Ames strain TA100. More specifically, the invention provides a high throughput alternative to the standard Ames mutation assay for the evaluation of the genotoxicity activity of compounds during an early stage of the drug development process. The invention also identifies the Upp locus as a mutational target that is capable of detecting a diverse spectrum of mutagenic events and further describes a S. typhimurium tester strain, designated FU100, (5-fluorouridine resistant) for use in the invention.

US20070166713 (Novartis AG) describes a system and method provided for performing genotoxicity screening. The system and method utilize: (1) one or more computers; (2) a frame grabber connected to the one or more computers; (3) a camera connected to the frame grabber; (4) a microscope connected to the one or more computers; (5) a slide feeder connected to the one or more computers; and (6) a program operating on the one or more computers. The program facilitates the screening of a second batch of biological material using a second genotoxicity testing method after screening a first batch of biological material using a first genotoxicity testing method. The screening operates substantially free of any manual manipulation of the camera, the microscope or the slide feeder.

US20070087364 (Massachusetts Institute of Technology) describes assays that can measure the genotoxic effects of compounds on hematopoietic cells such as erythroid cells in vitro, particularly human erythroid cells.

US20080138820 (GE Healthcare) describes compositions and methods, which provide an integrated approach to genotoxicity assessment procedures by employing a genetically engineered cell line to report on a number of key indicators of genotoxicity effects and mechanisms. Imaging and analysis of cells exposed to test agents allows automated analysis using high content cellular screening to identify cytostatic and/or cytotoxic activity, to quantify micronuclei formation, to discriminate aneugenic and clastogenic micronuclei and to detect DNA mutation and repair.

US20080096770 (Novartis) describes a rapid high throughput screening process to identify genotoxic compounds. This is accomplished by using a set of biomarker predictor genes that selectively screen for genotoxic or non-genotoxic compounds.

US20060110768 (Roche Palo Alto) relates to methods for determining genotoxicity in compounds, and test cells, transgenic animals, kits and reagents.

US20050221324 (Univ. Colorado) describes a genotoxicity test carried out using a genetic hybrid cell line, for example, a CHO cell line that contains human chromosome 11. This exemplified hybrid cell line expresses human CD59 on the cell surface, and the human CD59 gene serves as a test for mutagenic agents. The hybrid cell line is grown in the presence of a test compound, and the loss of cell surface CD59 is followed using a fluorescent-labelled antibody specific for human CD59 and flow cytometry to monitor the presence or absence of labelled antibody on particular cells. Absence of the labelled antibody on the surface of the cells is indicative of a mutation in the CD59 gene such that either no CD59 protein is made or there has been a mutation which results in the loss of the antibody binding site. A test compound, which causes CD59 loss, is deemed to be genotoxic (i.e., mutagenic). The mutations can be point mutations, deletions, inversions, insertions, or frame-shifts. The sensitivity of the assay is improved when the cells are first panned with antibody specific for the cell surface marker to remove spontaneous mutants prior to challenge with the test compound. Alternatively, or in addition, an antibiotic resistance marker is incorporated onto the same chromosome as the cell surface marker, and an antibiotic selection step precedes the challenge with the potential genotoxic agent.

EP1153137 (Phase 1 Molecular Toxicology) describes methods and kits for measuring mutant hypersensitivity assay using high throughput screening methodology to evaluate the mechanisms of toxicity of chemicals. The assay is performed in multi-well plates, such as those having 96 wells, making the process conducive to testing many compounds in a short period of time. The assay can test compounds for ability to cause, for example, DNA damage, ability to mutate genetic material (mutagenicity), the ability to cause cancer (carcinogenicity), cause protein or membrane damage, energy depletion, mitochondrial damage, as well as the more general genotoxicity.

GB2421730 (Astra Zeneca) describes a method for evaluating pharmacological target related toxicity in a mammal or in vitro based cell system wherein small interfering RNA (i RNA or RNAi or siRNA) is supplied to the mammal or cell to reduce target gene expression and detecting any change in at least one parameter of toxicity. The mammal may be a rat, mouse, guinea pig, hamster, pig, rabbit, dog, non-human primate or a human. The parameter of toxicity measured may include clinical observations, pathology, histopathology, chemistry, haematology, safety pharmacology or genotoxicity. The target may be a kinase, GPCR, ion channel or protease. Preferably the target is VEGFR-1, VEGFR-2 or VEGFR-3. Specific siRNA molecules are also claimed.

WO1996023895 (Univ. Texas) describes a method to detect and quantitatively assay the amount of DNA damage in large double-stranded DNA templates starting with less than 50 ng of sample DNA. This invention has utility in vivo for quantifying gene-specific DNA damage, measuring DNA repair rates and monitoring efficacy of anti-neoplasia therapy in patients. Additionally, this invention claims to be useful for detecting and assessing risk due to mutagenic environmental hazard and for monitoring hazard site dynamics. It includes a design for a device for accomplishing these objectives.

WO2007/002568 (Geron Corporation) describes a system for rapid determination of pharmacologic effects on target tissue types in cell populations cultured in vitro. The cells contain a promoter-reporter construct that reflects a toxicologic or metabolic change caused by the agent being screened.

US20080187927 describes methods for genotoxicity testing and geno-protection testing using genetic constructs having a hydroxyurea- and UV- and gamma radiation-induced promoter linked to a reporter gene, such as green fluorescent protein.

WO 98/44149 describes a genotoxic test, which concerns recombinant DNA molecules comprising a Saccharomyces cerevisiaie regulatory element that activates gene expression in response to DNA damage operatively linked to a DNA sequence that encodes a light emitting reporter protein, such as Green Fluorescent Protein (GFP). Such DNA molecules may be used to transform a yeast cell for use in a genotoxic test for detecting for the presence of an agent that causes or potentiate DNA damage. The cells may be subjected to an agent and the expression of the light emitting reporter protein (GFP) from the cell indicates that the agent causes DNA damage. The genotoxic tests described in WO 98/44149 detect the induction of repair activity that can prevent an endpoint being reached and therefore may be used to detect for the presence of DNA damaging agents.

U.S. Pat. No. 6,344,324 discloses a recombinant DNA molecule comprising the regulatory element of the hamster GADD153 upstream promoter region that activates gene expression in response to a wide range of cellular stress conditions, linked to a DNA sequence that encodes GFP. This reporter system is carried out in a human head and neck squamous-cell carcinoma cell line. However, problems associated with this reporter system are that it requires at least a four day treatment period at test agent concentrations that result in less than 10% cell survival, followed by analysis of fluorescence by flow cytometry. In addition, the biological relevance of any gene induction when tested with agents at this level of toxicity is debatable. Furthermore, this development does not disclose a means of specifically monitoring for the presence of agents that may cause or potentiate DNA damage, and the mechanism of GADD153 induction remains unclear. Hence, this system is of very limited use as a human DNA damage biosensor.

WO2005113802 (Gentronix) describes methods for detecting for the presence of an agent that putatively causes or potentiates DNA damage comprising subjecting a cell (containing a DNA sequence encoding a reporter protein operatively linked to a human GADD45α gene promoter and a human GADD45α gene regulatory element arranged to activate expression of the DNA sequence in response to DNA damage) to an agent; and monitoring the expression of the reporter protein from the cell. The invention also concerns expression cassettes, vectors and cells that may be used according to such a method and also modified media that may be employed in fluorescence assays and in preferred embodiments of the method of the invention.

WO2005012533 (Gentronix) describes a recombinant vector comprises a recombinant DNA molecule comprising a DNA sequence that encodes a light emitting reporter protein. The DNA sequence is operatively linked to a regulatory element arranged to activate expression of the DNA sequence in response to DNA damage. When used to transform a cell, the vector does not substantially alter the sensitivity of the cell to geneticin, when compared to the sensitivity of the cell, which has not been transformed with the vector.

U.S. Pat. No. 7,507,548 (University of Salamanca) describes a method for multidimensional detection of aberrant phenotypes in neoplastic cells to be used to monitor minimal disease levels using flow cytometry measurements

U.S. Pat. No. 7,358,059 (University of Salamanca) describes simultaneous quantification by flow cytometry of PIG-A associated proteins in red cells, platelets and leukocyte subsets using a single measurement

U.S. Pat. No. 6,287,791 (Becton Dickinson) describes a method for multidimensional cell differential analysis

Hastwell et al., 2006 (Mut. Res. 607 160-175) (Gentronix) describes a gene reporter system based on the human GADD45α gene. The recognition of GADD45α as a biomarker for genomic stress and damage has made it possible to engineer a reporter system in which the GADD45a promoter is fused to the cDNA encoding GFP. This has allowed the development of a 96-well microplate assay (GreenScreen HC) to identify genotoxins. However, this assay has a number of reported limitations (Olaharski et al., 2009 (Mut. Res., 672, 10-16).

US20080064053 (Walmsley & Billinton) describes a recombinant vector comprises a recombinant DNA molecule comprising a DNA sequence that encodes a light emitting reporter protein. The DNA sequence is operatively linked to a regulatory element arranged to activate expression of the DNA sequence in response to DNA damage. When used to transform a cell, the vector does not substantially alter the sensitivity of the cell to geneticin, when compared to the sensitivity of the cell, which has not been transformed with the vector.

Ohno et al., 2009 (Mut. Res. 656, 27-35) reported the construction of an alternative genotoxic luciferase gene reporter assay based on three tandem repeat sequences of the p53 response element from the p53R2 promoter (Ohno, et al., 2009, Mut. Res., 588, 47-57). One of the p53 target genes activated by genotoxic compounds is p53R2. The p53R2 gene product supplies nucleotides to repair damaged DNA (Tanaka et al, 2000, Nature, 404, 42-49; Xue et al, 2003, Cancer Res., 63, 980-986). Expression of p53R2 is known to be activated by γ-rays, UV light and genotoxic compounds in a p53-dependent manner.

Another approach for measuring mutations is based on the endogenous gene HPRT. This system has been applied successfully across several species. The assay is based on ex vivo mitogen stimulation of lymphocytes and resistance to the toxic agent, 6-thioguanine. It is technically demanding, subjective, time consuming and, in inexperienced hands, prone to errors.

Other mutation assays are based upon measuring transgene activity (e.g. lacZ and lacl) but limitations also exist for these methods, in that it is non-generic and requires the generation of specific transgenic cell lines or animals.

US 2007/0274919 (Litron) describes a method for the enumeration of in vivo gene mutation using animal models. The method utilizes differential staining of GPI-anchor deficient erythrocyte populations to distinguish between wild-type and PigA gene mutants. Quantitative analyses can be conducted on erythrocytes and/or reticulocytes, and is based upon fluorescent emission and light scatter following exposure to an excitatory light source. Counting of mutant erythrocytes or reticulcoytes relative to the number of total erythrocytes or reticulocytes can be used to assess the DNA-damaging potential of an exogenous chemical agent, the DNA-damaging potential of an exogenous physical agent, the effects of an exogenous agent which can modify endogenously-induced DNA damage, and the effects of an exogenous agent which can modify exogenously-induced DNA damage. Kits for practicing the invention are also disclosed. The application describes an animal, in vivo-based method. A limitation of this approach is that the erythrocytes and reticulocytes must be separated from other blood components prior to exposure to the anti-CD24-antibody and flow cytometric analyses and is therefore not convenient.

Agents that specifically bind to GPI-anchored proteins have been investigated as diagnostic probes for PNH. One such example is aerolysin from the gram-negative bacterium Aeromonas hydrophila (Brodsky et al, 2000, Am.J. Clin. Pathol, 114, 459-466; Krauss, 2003, Ann. Clin. Lab. Sci, 33, 401-406). A non-toxic aerolysin mutant conjugated with a fluorescent dye, FLAER, is commercially available (Protox Biotech, British Columbia, Canada), (Sutherland et al Cytometry, 2007, 72B, 167-77) and can detect PNH cells as effectively as monoclonal antibodies against CD55 and CD59.

Alpha toxin, which is secreted from the gram-positive bacterium Clostridium septucum also specifically binds GPI-anchored proteins on the cell surface. An enhanced green fluorescent protein-alpha protein fusion protein has been used to diagnose PNH by flow cytometry (Shin et al, 2006, J. Mol. Microbiol. Biotechnol., 11, 20-27).

Miura et al. 2008, (Environ. Molecular Mutagenesis, 49, 614-621) described the development of an in vivo gene mutation assay using the endogenous PigA gene. This involved conditions specifically-designed for the flow cytometric identification of GPI-deficient cells among the peripheral red blood cells (rbcs) and spleenic T-cells of rats. These were used for the determination of spontaneous and induced frequencies of GPI-deficient cells in control and N-ethyl-N-nitrosourea-treated rats. The GPI-anchored cell surface markers CD48 and CD59 were used as reporters of PigA mutation in peripheral rbcs and spleenic T-cells, respectively, In addition, a fluorescent reagent specifically binding GPI anchors (proaerolysin; FLAER) was used to assess the frequency of GPI-deficient rbcs.

Dobrovolsky et al. (2009) (Environ Mol Mutagen, 50, 7, 515-590, Environmental Mutagen Society meeting abstracts) describes a flow cytometric detection of Pig-A mutant red blood cells using an erythroid-specific antibody with specific applications of the method for evaluating the in vivo genotoxicity of methylphenidate in adolescent rats.

Miura et al., 2008 (Environ. Molecular Mutagenesis, 49, 622-630) continued with the description of an in vivo gene mutation assay using the endogenous PigA gene. In this study, GP1-deficient rbcs and spleenic T-cells from rats were quantified by examining the mutational basis of the phenotype. This group developed a method for the selection and expansion of GP1-deficient rat spleenic T-cells using 2 nM proaerolysin and examined: (i) whether the proaerolysin-resistant cells lack tissue-specific GP1-anchored protein markers on their surface; and (ii) whether these GPI-deficient cells contain mutations in the PigA gene.

WO 02/040994 (Yissum Research Development Company) relates to an in vivo method for evaluating the effect of test drugs on the nervous system of animals.

WO99136778 (University of Victoria) elates to toxins that bind to GPI anchors or GPI anchored proteins together with a method which is used for the diagnosis of Paroxysmal Nocturnal Hemoglobinurea whereby a blood sample is taken and is used for further analysis.

Ogura et al. (2001) IUBMB Life 51: 381-385: describes urokinase activator receptor expression in human gingival fibroblasts. Plasminogen activator activity, released by phosphatidylinositol-specific phospholipase C, which detaches the GPI anchor, was reported to be increased by interleukin-1β.

Jarvis et al. (1997) Int. J. Cancer 71: 1049-1055 relates to the expression and function of CD59 in human prostate cancer tissue. Fluorescence assays were carried out by flow cytometric analysis.

Zhao et al. (2009) Oncol. Reports 21:1405-1411 relates to the expression of CD55 and CD59 expression in lung squamous carcinoma cells and their clinical significance.

Ruiz et al. (2006) Transplant. Proc. 38: 1750-1752 describes CD55 and CD59 deficiency in transplant patient populations with a possible association with

Paroxysmal Nocturnal Hemoglobinurea-like symptoms in Campath-treated individuals. CD55 and CD59 were measured on peripheral blood leucocytes by flow cytometry techniques.

Song et al. (2007) Leukemia Research 31: 1701-1707 describes the role of glycosylphosphatidylinositol-specific phospholipase D in the homing of umbilical cord blood, mobilized peripheral blood and bone-marrow derived hematopoietic stem/progenitor cells. The expression of GPI-anchored proteins (CD48 and CD90) on the cells was analysed by flow cytometry.

Ellery et al. (2008) Clin Appl. Thromb/Haemostatsis 14: 267-278 describes heparin-releasable and glycosylphosphatidylinositol-lipid and forms of tissue factor pathway inhibitor (TFPI). A twenty-fold increase of TFPI following phospholipase C treatment of human umbilical vein endothelial cells was observed. TFPI activity was measured using a coupled enzyme assay using a chromogenic substrate.

Bannerji et al. (2000) Blood 96: 164a relates to the cell surface complement inhibitors CD55 and CD59 and how these factors may mediate chronic lymphocytic leukemia resistance in Rituximab treated patients. Antibodies were used to quantitate CD55 and CD59.

Thus, genotoxic agents are known to cause damage to the genetic material of cells and are therefore potentially mutagenic or carcinogenic. Although a number of assays for measuring mutation rates in vitro and in vivo have been developed, improved methods are needed for understanding the risk and safety of such agents.

In particular, there is a need for in vitro methods which can utilise human cells and cell lines grown in culture rather than being restricted to the use of ex vivo cells, such as erythrocytes and reticulocytes. Furthermore, there is a desire to use assays which closely reflect the in vivo condition.

### SUMMARY OF THE INVENTION

The present invention describes methods for measuring the activity of genotoxic agents and is based on the generation of mutations within the phosphatidyl inositol glycan group A, (PigA) gene locus.

In a first aspect of the present invention, there is provided an in vitro method of measuring the genotoxicity of an agent in human cells comprising the steps of:
(i) providing an in vitro culture of human cells comprising the phosphatidylinositol glycan biosynthesis class A (PigA) gene and expressing at least one glycosylphosphatidylinositol (GPI) anchor;
(ii) determining a control level of said GPI-anchor or a GPI-anchored protein in said cells;
(iii) exposing the cells to an agent;
(iv) determining a treatment level of the GPI-anchor or said GPI-anchored protein;
(v) comparing said treatment level with said control level of the GPI-anchor or the GPI-anchored protein,
   wherein a difference in the levels of the GPI-anchor or the GPI-anchored protein is indicative of the genotoxicity of said agent.

The method is based upon either the absence or reduction of GPI anchor and/or GPI-anchored proteins at the cell surface caused by mutations at the PigA gene locus.

In one aspect, the levels of the GPI anchor are determined using a GPI- anchor-binding moiety.

In another aspect, the GPI-anchor binding moiety is selected from the group consisting of antibody, proaerolysin, aerolysin and alphatoxin.

In one aspect, the GPI anchored protein is selected from the group consisting of CD14, CD16, CD24, CD28, CD48, CDw52, CD55, CD56, CD58, CD59, CD66a, CD66c, CD66d, CD66E, CD67, CD73, CD87, CD90, CD108, CD157, acetylcholinesterase (AchE), alkaline phosphatase (NAP), Urokinase type plasminogen activating receptor (uPAR), JMH protein, GDNFR, CNTFR, TAG-1, PrP, Glypican protein, Semaphorin 7, CEA, GFR, Ly6G, transferrin receptor, Contactin (F3) and T-cadherin.

In another aspect, the GPI anchored protein is selected from the group consisting of CD55, CD59, acetylcholinesterase (AchE) and alkaline phosphatase (NAP).

In yet another aspect, the cells are selected from the group consisting of hepatocytes, cardiomyoctyes, blood cells, haematopoietic cells, stem cells, neuronal cells, renal cells, placental cells, osteoblasts, osteocytes, osteoclasts, spleen cells, pancreatic cells dermal cells and cells isolated from the gut.

In a further aspect, the cells are selected from a group of cell lines consisting of human monocytic leukaemia cell line, hepatoma cell line Hep3B, hepatoma cell line HepG2, erythroleukemia tumor cell line K562, CHO, Hela and NTera-2 human pluripotent embryonal carcinoma cell line.

In one aspect, the levels of GPI anchored protein are determined by quantitative immunocytochemistry.

In another aspect, the levels of GPI anchored protein are determined by Surface Plasmon Resonance (SPR).

In a further aspect, the levels of GPI anchored protein are determined by measurement of GPI anchored protein enzymatic activity. In a preferred aspect, the enzymatic activity is acetylcholinesterase or alkaline phosphatase activity.

In one aspect, the GPI anchored protein is released from the cells prior to determining the level of GPI anchored protein. Preferably, the GPI anchored protein is released from the cells by treatment with phospholipase C.

In another aspect, the method is a multiplex method which additionally comprises measuring a cellular event which is unrelated to the levels of GPI anchor or GPI anchored protein. For example, the cellular event is selected from the group consisting of cell viability, micro-nuclei presence, mitochondrial membrane integrity, cell proliferation and apoptosis.

In a further aspect, the agent is a chemical compound. Examples of chemical compounds which can be tested in the method of the invention include radio-isotopes, drug candidates, pharmaceuticals, agrochemicals, pesticides, environmental agents, food additives and cosmetics.

In another aspect, the agent is an electromagnetic radiation. Suitable forms of electromagnetic radiation include X-ray, microwave, UV and IR radiation.

In a second aspect of the present invention, there is provided the use of the method of the first aspect for high content screening.

In a third aspect of the present invention, there is provided a kit comprising a reagent capable of quantifying the level of a GPI anchor or a GPI anchored protein and instructions for carrying out the method. Preferably, the reagent is a labelled antibody to a GPI anchor or GPI anchored protein or an enzyme substrate of a GPI anchored protein.

Further features and advantages of the invention will become apparent from the following description of preferred embodiments of the invention, given by way of example only, which is made with reference to the accompanying drawing.

Definitions

The term “genotoxicity” as related herein describes a deleterious action of an agent on a cell's genetic material affecting its integrity. Genotoxic agents are known to be potentially mutagenic or carcinogenic, specifically those capable of causing genetic mutation and of contributing to the development of tumours.

A “glycosylphosphatidylinositol (GPI)-anchor” is a glycolipid that may be attached to the C-terminus of a protein during cellular biosysnthesis of the protein. A GPI anchor is composed of a phosphatidylinositol group that may be linked through a carbohydrate containing linker (glucosamine and mannose glycosidically bound to the inositol residue) to the C-terminal amino acid of a mature protein.

A “glycosylphosphatidylinositol (GPI) anchored protein” is a protein tethered or linked or attached to the cell surface through the GPI-anchor.

The term “mutagen” as related herein describes a physical or chemical agent that changes the genetic material of an organism and thus increases the frequency of mutations above the natural background level. As many mutations cause cancer, mutagens are typically also carcinogens.

The term “mutations” as related herein describe changes to the nucleotide sequence of the genetic material of an organism. Mutations can be caused by copying errors in the genetic material during cell division, by exposure to ultraviolet light, or ionizing radiation, chemical mutagens, or viruses. Changes in DNA caused by mutation can cause errors in protein sequence, creating partially or completely non-functional proteins. To function correctly, each cell depends on thousands of proteins to function in the right places at the right times. When a mutation alters a protein that plays a critical role in the body, a medical condition can result. A condition caused by mutations in one or more genes is called a genetic disorder.

As disclosed herein, the term “vital dye” relates to stains and dyes frequently used in biology and medicine to highlight structures in biological tissues or cells for viewing, typically with the aid of a microscope.

As disclosed herein, the term “multiplex assay” or “multiplex method” relates to or is a method of measurement or communication of information or signals from two or more messages from the same source (an example of a muliplex assay is described by Ugozzoli, et al. 2002 (Anal. Biochem., 307, 47-53).

The term “antibody” as described herein or is used to include intact antibodies and binding fragments thereof. Typically, fragments compete with the intact antibody from which they were derived for specific binding to an antigen fragment including separate heavy chains, light chains Fab, Fab′ F(ab′)₂, Fabc, and Fv. Fragments are produced by recombinant DNA techniques, or by enzymatic or chemical separation of intact immunoglobulins. The term “antibody” also includes one or more immunoglobulin chains that are chemically conjugated to or expressed as fusion proteins with other proteins. The term “antibody” also includes bispecific antibodies. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, for example, Songsivilai & Lachmann, 1990, Clin. Exp. Immunol., 79, 315-21; Kostelny et al., 1992, J. Immunol. 148:1547-53.

An “antigen” is an entity to which an antibody specifically binds.

The term “immunocytochemistry” as used herein is a laboratory method which uses antibodies that target specific peptides or protein antigens via specific epitopes. These bound antibodies can then be detected using one of several methods, but often by cellular imaging. Immunocytochemistry allows researchers to evaluate whether or not cells in a particular sample express the antigen in question. In cases where an immunopositive signal is found, immunocytochemistry also allows researchers to determine which cellular compartment is expressing the antigen.

The term, “high-content screening”, as used herein, is a drug discovery method that uses living cells as the test tube for molecular discovery. It describes the use of spatially or temporally resolved methods to discover more from an individual experiment than one single experiment with one output alone. It uses a combination of cell biology, with molecular tools, typically with automated high resolution microscopy and robotic handling (Giuliano et al., 1997, J Biomol Screen., 2, 249-259). The method described herein describes use of an assay method with living cells. The present invention therefore provides an integrated high-content, high-throughput, information-rich cell-based assay method involving the PigA gene and GPI-linked proteins, capable of yielding data on biological activity of exogenous agents or toxins acting upon living cells grown in culture.

### BRIEF DESCRIPTION OF THE DRAWINGS

The following description, given by way of example, is not intended to limit the present invention to any specific embodiment described. The description may be understood in conjunction with the accompanying figure.

FIG. 1 is a schematic diagram illustrating a GPI anchor and a GPI anchored protein.

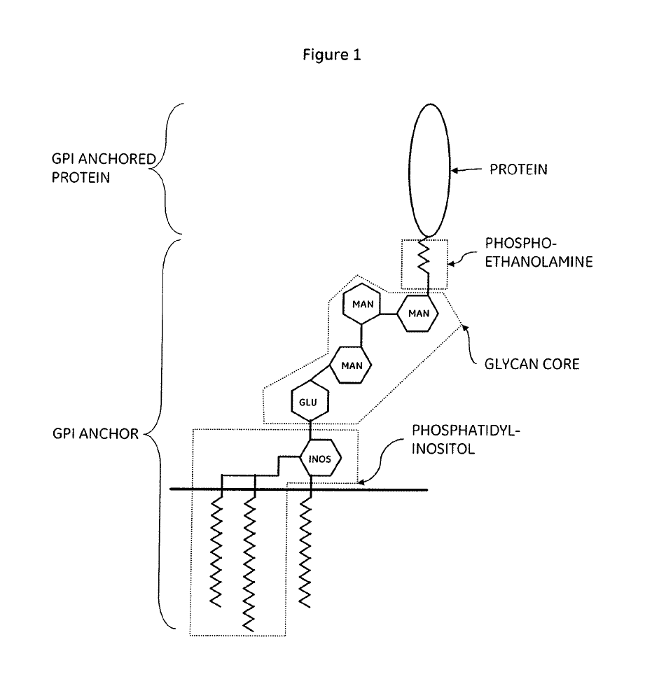

### DETAILED DESCRIPTION OF THE INVENTION

The PigA gene encodes a protein required for the biosynthesis of N-acetylglucosaminyl phosphatidylinositol (GlcNAc-PI), the first intermediate of the glycosylphosphatidylinositol (GPI) biosynthetic pathway. GPI consists of multiple components and its biosynthesis involves multiple gene products. The GPI moiety is found in the majority of cells in which it serves to attached GPI-anchored proteins to the cell surface.

The actual GPI anchor is attached to the C-terminus of a protein during post-translational modification. It is composed of a phosphatidylinositol (PI) group linked through a carbohydrate linker (glucosamine and mannose). The two fatty acids within the hydrophobic PI group anchor the protein to the cell membrane. Proteins destined for GPI-anchoring contain a signal peptide, thus directing them into the ER. These proteins possess hydrophobic amino acids at the C-terminus ensuring their retention in the ER membrane. The hydrophobic moeities are eventually cleaved and replaced by the GPI-anchor. As the protein progresses through the secretory pathway, it is transferred via vesicles to the Golgi apparatus and finally to the extracellular surface where it remains attached to the exterior of the cell membrane.

In mammals, >200 cell surface proteins are anchored to the membrane via attachment to a GPI moiety. These proteins include enzymes and receptors and are potential targets for measuring both the in-vitro and in-vivo generation of mutations induced by genotoxic chemicals at the PigA locus.

Of all the genes involved in generating the GPI-anchor moeity only PigA is located on the X-chromosome. This is a highly desirable feature, since only one functional copy is present. Therefore only a single allele is available for mutation and this should facilitate disruption the PigA gene locus and the generation of phenotypes which can be readily detected. Hence a mutation that results in the generation of a non-functional PigA gene product should result in the absence of GPI-anchored proteins located at the plasma membrane surface.

The PigA gene locus possesses a high degree of inter-species conservation and somatic mutations within the locus are linked to Paroxysmal Nocturnal Hemoglobinuria (PNH), (Norris et al., 1994, Blood, 83, 816-821 and Nishimura et al., 1999, Am. J. Hematol., 62, 175-182). In this disease, decay accelerating factor (DAF or CD55) and CD59 which are normally linked to the cell surface via a GPI-linkage are absent from the surface of red blood cells. These cells because of a mutation in the PigA gene are deficient in the GlcNAc-PI moiety. This deficiency results in the absence of GPI-linked proteins on the cell surface.

PNH is an acquired hematologic disorder characterized by the clonal expansion of affected cells derived from various hematopoietic lineages. These are derived from an abnormal multi-potent hematopoietic stem cell and by complement-mediated hemolysis of cells lacking CD55 and CD59 at the cell surface (Rosse, W. F. Blood Rev., 3, 192-200, 1989). Therefore PNH is an acquired genetic disorder. The mutated cell clone endows all its descendants with the mutated gene. This includes, for example, red cells, leukocytes and platelets. These mutant cells originate and expand side-by-side with normal cells creating a mosaic in which the proportion of abnormal erythrocytes in the blood determines the severity of the disease. Its clinical hallmark is black urine on arising from sleep (Mahoney, J. F..et al., Blood, 79: 1400-1403, 1992).

Several approaches are available to detect and quantify the presence of GPI anchors/GPI anchored proteins expressed from the PigA gene:

Antibody Mediated Detection of GPI Anchored Proteins.

Specific antibodies targeted against GPI-anchored proteins (see Table 1 and 2) are tagged with enzyme reporter groups such as luciferase, β-galactosidase and horseradish peroxidase. These can be used in combination with commercially available luminescent and fluorescent substrates thereby generating sensitive assays. Any mutations at the PigA gene locus that reduces the presence of GPI-anchored proteins at the cell surface will be represented by a reduction in enzymatic activity.

Alternatively fluorescent reporters such as Cy5 or Cy3 (GE Healthcare) may be used to generate specific fluorescently-tagged antibodies against specific GPI-linked proteins. These reagents will facilitate a multiplexed quantitative immunocytochemistry and high content analysis (HCA) approach for use on optical imaging instrumentation. The method could involve the staining of cells with the nuclear dyes Hoechst and propidium iodide. Calcein AM could also be incorporated into the analysis in combination with propidium iodide as live-dead cellular reporters by measuring the decrease in propidium iodide staining and the increase in Calcein staining, following induction of mutations (Chen. et al., 2001, Cancer Research, 61, 654-658). A multiplex HCA approach could also be correlated with other cytotoxic markers such as cell viability (using the CellTiter-Glo® Luminescent and CellTiter-Fluor Cell Viability Assays, Promega), micro-nuclei analysis (using the InCell 2000 Micronuclei Analysis Module, GE Healthcare), mitochondrial membrane integrity (using the HCS Mitochondrial Health Kit, Invitrogen).

GPI-linked analytes may also be detected via surface plasmon resonance (SPR) using specific immobilised antibodies. The presence of GPI-linked molecules can be measured either on the cell surface, in cellular extracts or in a soluble form in cell culture supernatants.

Detection of Functional Activity of GPI Anchored Proteins.

Suitable methods include the direct measurement of endogenous enzymatically active GPI-anchored proteins such as acetylcholinesterase and alkaline phosphatase. This can be achieved using fluorescent or luminescent substrates (e.g. acetylcholine/ATP and dioxetane-based substrates). The Vector Red substrate for alkaline phosphatase can also be used for cell or tissue staining and for use in HCA optical imaging

As an alternative to measurement of functional activity in-situ, GPI anchored proteins may be released from cells for assay in solution. Phospholipase C (PLC) is an enzyme that is known to cleave the phospho-glycerol bond found in GPI-anchored proteins thereby releasing the attached protein from the membrane associated GPI anchor. This property of PLC can be used in vitro to liberate the protein into the cell medium. Simple assays can then be performed using aliquots of the cell culture medium. In addition, chaotrophic agents, will also cause the release of GPI-linked proteins from the outer cell membrane.

Direct Detection of GPI Anchors

It is known that the bacterial toxins aerolysin and Clostridium septicum alpha-toxin form channels in the membranes of target cells by binding to GPI anchors.

Enzymatically or fluorescently-labelled non-toxic derivatives of these toxins, for example pro-aerolysin derived from Aeromonas hydrophila (FLAER reagent: Protox Biotech, British Columbia, Canada; Sutherland, et al., 2007, Cytometry, 72B, 167-77) or alpha toxin (Shin, D. J. et al., 2006, J. Mol. Micro. & Biotech. 11, 20-27) may be used to detect and measure the presence of the GPI moiety directly. This approach has the advantage of accessing the GPI moiety directly facilitating generic assays which are independent of the attached protein moiety. Such assays are likely to be more sensitive in detecting genotox damage since any decrease in GPI anchor levels are detected, whereas antibody based approaches measure only decreases in GPI anchor expression associated with the protein recognised by the antibody in use.

Direct Detection of PigA Gene Expression

Quantitative RT-PCR may be used to monitor mutations within the PigA gene promoter region that cause changes in the mRNA expression levels generated in response to the exposure of cells to genotoxic compounds.

Thus, the present invention identifies procedures that can be employed as simple in vivo or in vitro gene mutation assays that can be used to evaluate target compounds (e.g. drug, chemical, cosmetic or physical agents) for genetic toxicity. In addition, the methods described here are likely to be fast, reliable and accurate and can be carried out without the need for complex transgenic animals or cells. Indeed, the antibody-luminescent enzyme based approach, will have major advantages over previous gene mutation assay methods, in terms of signal magnitude and ease-of-use.

### EXAMPLES

The invention is illustrated by reference to the following examples. For purposes of illustration the monitoring and quantification for the presence of the well-characterized GPI-anchored proteins CD55 and CD59 will be described. However, while preferred illustrative embodiments of the present invention are described, one skilled in the art will appreciate that the present invention can be practised by other than the described embodiments, which are presented for purposes of illustration only and not by way of limitation.

### Examples of Cell Types

The human mono-cytic leukaemia cell line THP-1 expresses many “cluster of differentiation molecules” including CD55 and CD59 (Yokochi, S., et al., 2001, J.

Interferon Cytokine Res., 21, 389-398). Therefore treatment with genotoxic compounds and the subsequent monitoring for the presence of the GPI-linked proteins CD55 and CD59 will facilitate the assessment and measuring of mutation rates at the PigA gene locus. THP-1 cells are available from ATCC (Cat. no. TIB-202) and are propagated as a suspension culture in RPMI-1640 medium supplemented with 0.05 mM 2-mercaptoethanol and 10% foetal bovine serum, in 5% CO₂ at 37.0° C. Cultures are maintained at 2-4×10⁵ viable cells per ml and should be sub-cultured at 8×10⁵ cells per ml.

Primary hepatocytes also express CD55 and CD59 albeit at low basal levels (Halme, J. 2009, Primary human hepatocytes are protected against complement by multiple regulators. Mol. Immunol. May 13, Epub ahead of print). CD55 and CD59 expression was enhanced by IFNγ, IL-1β, TNFα and IL-1β, IL-6 and TNFα respectively. Primary hepatocytes and cell culture medium are available from Lonza (Cat. no. CC-2703T75 and CC-3198 respectively).

In addition, the Hepatoma cell lines Hep3B and HepG2 (ECACC cat. no. 86062703 and 85011430 respectively) also express CD55 and CD59 and that a combination of TNFα, IL-1β, and IL-6 was shown to increase their expression levels (Spiller, O. B. et al., 2000, Clin. Exp. Immunol. 121, 234-241). Hep3B and HepG2 are propagated in Dulbecco's Modified Eagle's Medium supplemented with 10% foetal calf serum and 2 mM glutamine. Therefore hepatocytes can be used to monitor the genotoxicity of both parent drug compounds and their metabolites.

Many commonly used cell lines express CD55 and CD59, for example the human erythroleukemia tumor cell line K562 (Jurianz, K. et al., 2001, Int. J. Cancer, 93, 848-854). These suspension cells are available from ATCC (Cat. no. CCL-243) and required Iscove's Modified Dulbecco's Medium supplemented with 10% fetal bovine serum. New cultures are seeded at 1×10⁵ cells/ml and sub-cultured at 1×10⁶ cells/ml.

No studies describe the expression of CD55 and CD59 by CHO and Hela cells. However, both cell lines express many GPI-anchored proteins (Shin, D. J., et al., 2006, Mol. Microbiol. Biotechnol. 11, 20-27). CHO cells are available from ECACC (Cat. No. 85050302) and are cultured in Ham's F12 supplemented with 2 mM Glutamine and 10% foetal Bovine Serum. Hela cells are available from ATCC (Cat. No. CCL-2) and are cultured in Eagle's Minimum Essential Medium supplemented with 10% foetal bovine serum.

NTera-2 cells are human pluripotent embryonal carcinoma and are available from ECACC (Cat. no. 01071221). These cells have been shown to express the GPI-anchored enzymes acetylcholinesterase and alkaline phosphatase before and after retinoic acid mediated neuronal differentiation (Llanes, C. et al., 1995, J. Neurosci. Res. 42, 791-802 and Draper, J. S. 2002, J. Anat. 200, 249-58 respectively). Therefore a direct activity measurement for the presence of either these GPI-linked enzymes could be utilised to assess mutations at the PigA locus. For example NTERA-2 cells would be treated with a range of genotoxic compounds and acetylcholinesterase and alkaline phosphatise enzymatic activities measured directly using commercially available bioluminescent substrates.

Therefore by monitoring the presence or activity of specific GPI-anchored proteins, all of the cell types described above can be potentially used for in-vitro cellular assays designed to measure the effect of any genotoxic compound at the PigA locus.

CD55, CD59 Antibodies and GPI-Binding Proteins

Examples of specific commercially available antibodies targeted against a range of GPI-linked proteins are described in Table 2. Any one of these proteins in combination with the specific antibody is suitable for monitoring the presence of a functional GPI-moiety and thereby a functional PigA gene locus.

TABLE 2
GPI-anchored proteins and details of commercially-available antibodies. All are
potential targets for monitoring the effect of genotoxic agents at the PigA gene locus.

| CD Molecule | Function | Antibody details |
|---|---|---|
| CD14 | LPS receptor | Abcam (MEM-18, ab8103) |
| CD16 | Low affinity receptor for IgG | Abcam (MEM-154, ab664) |
| CD24 | Signal transducing molecule | Abcam (M1/69, ab64064) |
| CD48 | Adhesion molecule | Abcam (MEM-102, ab9185) |
| CDw52 | Surface antigen on T and B cells | Abcam (HI186, ab2576) |
| CD55 | Decay accelerating factor | Abcam (67, ab20145) |
| CD56 | NCAM-120 | Abcam (RNL-1, ab9018) |
| CD58 | LFA3 Adhesion molecule | Abcam (B-L28, ab47070) |
| CD59 | Inhibitor of complement attack complex | Abcam (MEM-43, ab9182) |
| | formation | |
| CD66acd & e | CEACAM family granulocyte specific | Abcam (YTH71.3, ab23982) |
| CD67 | mmunoglobulin, CEA superfamily member | Abcam (80H3, ab19779) |
| CD73 | Ecto-5′-nucleotidase | Abcam (7G2, ab54217) |
| CD87 | uPA Receptor | Abcam (VIM-5, ab63470) |
| CD90 | Thy-1 (T-cell antigen) | Abcam (MRC OX-7, ab225) |
| CD108 | Semaphorin 7a (JMH Blood group antigen) | Abcam (MEM-150, ab8222) |
| CD157 | Bone marrow stromal cell antigen 1 | Abcam (ab74301) |
| AChE | Acetylcholinesterase | Abcam (HYB 111-05, ab23455) |
| NAP | Alkaline phosphatase | Abcam (4H1, ab54778) |
| uPAR | Urokinase-type plasminogen | Abcam (PGM2005, ab8473) |
| GDNFR | glial cell line-derived neurotrophic factor | Receptor 1 - Abcam (ab8026) |
| | receptor | Receptor 2 - Abcam (ab8027) |
| | CNTFR | Abcam (ab58560) |
| TAG-1 | Transient axonal glycoprotein 1 | Abcam (ab68945) |
| PrP | Prion protein | Abcam (1E4, ab51574) |
| Glypican | GPI-anchored proteoglycans | Glypican 1 - Abcam (ab55971) |
| family | | Glypican 3 - Abcam (ab60124) |
| | | Glypican 4 - Abcam (ab77534) |
| | | Glypican 5 - Abcam (ab67655) |
| | | Glypican 6 - Abcam (ab71343) |
| Semaphorin | Semaphorin family include secreted, | Semaphorin 7a - Abcam (MEM- |
| | transmembrane, and GPI anchored | 150, ab26012) pre-labelled with |
| | extracellular molecules that are involved in | FITC. |
| | regulating axon guidance. Class 7 are GPI- | |
| | anchored. | |
| CEA | Carcino Embryonic Antigen is synthesised | CEA - Abcam (CI-P83-1, |
| | during development in the foetal gut, | ab46538) pre-labelled with FITC |
| GFR | Members of the glial cell line-derived | GFR alpha 3 antibody (ab8028) |
| | neurotrophic factor (GDNF) receptor family. | |
| Ly6G | Also known as the myeloid differentiation | Abcam (RB6-8C5, ab25024) |
| | antigen Gr1, is expressed on monocytes in | pre-labelled with FITC |
| | the bone marrow. | |
| Transferrin | Cellular uptake of iron occurs via receptor | Abcam - (B349, DF1513, |
| receptor | mediated endocytosis of ligand occupied | ab8598) |
| | transferrin receptor into specialized | |
| | endosomes. | |
| Contactin | Contactins mediate cell surface interactions | Contactin 1 - Abcam (ab68941) |
| family | during the development of the nervous | Contactin 3 - Abcam (ab68961) |
| | system | Contactin 6 - Abcam (ab69014) |
| H Cadherin | Cadherins are calcium dependent cell | Abcam - (ab36905) |
| (also known | adhesion proteins. | |
| as T and 13) | | |

Monoclonal antibody against CD55 and CD59 are available from Abcam (Cat. no. 67, ab20145 and MEM-43, ab9182 respectively). These can be coupled to enzyme reporter groups such as luciferase, beta-galactosidase and fluorescent moieties such as cyanine dyes (GE Healthcare).

In addition, the labeling of the GPI-binding proteins pro-aerolysin (Sutherland, et al., 2007, Cytometry, 72B, 167-77) or the Clostridium septicum alpha toxin (Shin, D. J. et al., 2006, J. Mol. Micro. & Biotech. 11, 20-27) with functionally active enzymes will facilitate the direct detection and quantitation of the GPI moiety.

Conjugation of Antibodies to Specific Enzyme Activities

The conjugation of an antibody to luciferase was described by Jablonski, E. 1985, Anal. Biochem., 148, 199-206. Briefly, this involved modifying luciferase with the reagent S-acetylmercapthsuccinic anhydride in order to provide accessible sulfhydydryl groups for the subsequent reaction with maleimide residues on the derivatised antibody.

S-acetylmercapthsuccinic anhydride (2 ,moles in N,N-dimethyformamide was added to 150 nmol luciferase in 0.1M potassium phosphate pH 7.0 buffer. The reaction mixture was incubated at room temperature for 40 min, applied to a G25 Sephadex column to removed un-reacted agents. The derivatised enzyme was subsequently eluted using 0.1M potassium phosphate pH 7.0 buffer and stored frozen in the acetylated form.

The antibody (1 mg/ml) was derivatised in PBS with a 50-fold molar excess of meta-maleimidobenzoyl-N-hydroxysuccinimade ester (MBS) (0.8 mg) in 10 μl dioxane. This mixture was incubated for 30 min at 30° C. and then applied to a Sephadex G-25 column and eluted with 0.1 M potassium phosphate pH 7.0.

Conjugation was achieved by firstly, removing the acetyl groups from the luciferase by treatment with hydroxylamine (5% final volume) for 3 min at room temperature. The thiolated luciferase (32 nmol) was reacted with the derivatised antibody (8 nmol) with 13 mM FMN. The reaction mixture was dialysis overnight against 0.1M potassium phosphate pH 7.0 and the effectiveness of the conjugation reaction was characterised in terms of mobility on by SDS-PAGE and functional immunoassays.

Beta-galactosidase/antibody conjugation was described by O'Sullivan, M. J. et al., 1979 (Anal. Biochem. 100, 100-108) following two distinct procedures. Firstly, the antibody was suspended in 50 mM N-ethylmorpholine-HCl pH 7.5 to which a 0.1 ml solution of methyl-mercaptobutyrimidate (10 mg per ml) in 0.1 M Na₂CO₃ was added. The mixture was incubated for 1 hour at room temperature. Sepharose—anti IgG column was used to immobilize the antibody and to wash out any un-reacted reagents. The modified antibody was eluted with HCl pH 2.5 into 50 mM acetate buffer pH 6.0. Saturated N,N′-o-phenylenedimaleimide in 0.1 M acetate buffer was added to the antibody solution and incubated for 30 min at 30° C. Excess N,N′-o-phenylenedimaleimide was removed by applying the mixture to a Sephadex G-25 column.

The derivatised antibody was mixed with 13-galactosidase (2 mg) and incubated for 30° C. for 1 hr. The conjugation reaction was terminated by the addition of β-mercaptoethanol to a final concentration of 10 mM and the mixture dialyzed overnight at 4° C. against 10 mM Tris-HCl pH 7.5 containing 10mM MgCl₂ and 10mM β-mercaptoethanol. The β-galactosidase conjugated antibody was purified using DEAE chromatography and a linear NaCl gradient (50-200 mM).

The alternative method described by O'Sullivan, M. J. et al., 1979 involved adding MBS (0.32 mg) in dioxane to the antibody (1 mg) solution in 0.1 M phosphate buffer pH 7.0 containing 50 mM NaCl. The reaction was incubated for 30° C. for 1 hr. The mixture was applied to a Sephadex G-25 column and eluted with 0.1 mM phosphate buffer pH 7.0 containing 10 mM MgCl₂ and 50 mM NaCl. The β-galactosidase was immediately mixed with the eluted antibody and incubated at 30° C. for 1 hr. The reaction was terminated by the addition of β-mercaptoethanol (final concentration of 10 mM). The β-galactosidase conjugated antibody was applied to a DEAE-agarose column and eluted (after extensive washing with 10 mM Tris-HCl pH 7.0 containing 10 mM MgCl₂ and 50 mM NaCl) in 10 mM Tris-HCl pH 7.0 supplemented with 0.2 M NaCl. The β-galactosidase conjugated antibodies generated by either of these methods were characterized in terms of enzyme activity and functional immunoassays.

A recent method for generating 13-galactosidase conjugated antibodies is described by Liu, Z. et al., 2000, Immunological Methods, 234, 153-167. These authors used the water soluble hetero-functional cross-linker sulfosuccinimidyl 4-[-N-maleimidomethyl]-cyclohexane-1-carboxylate (sulfo-SMCC). The antibody was suspended in 100 mM phosphate buffer pH 7 at 5 mg /ml. The maleimide-activated antibody was prepared by adding 1 mg sulfo-SMCC to the antibody (1.5 mg). The mixture was incubated for 30 min at 30° C. Excess un-reacted sulfo-SMCC was removed by Sephadex G-25 chromotagraphy. The activated antibody was eluted using 100 mM phosphate buffer pH 7 and 150 mM NaCl and the antibody was subsequently used at 2 mg/ml.

The antibody-enzyme conjugate was generated by mixing the maleimide-activated antibody (1 mg) with 0.6 mg β-galactosidase in 100 mM phosphate buffer pH 7 supplemented with 150 mM NaCl. The mixture was incubated for 30° C. for 40 min. The reaction was terminated with β-mercaptoethanol (final concentration of 10 μM). The β-galactosidase conjugated antibody was purified using Sephadex G-200 chromatography and functionally tested by a cell-based ELISA method.

Comprehensive descriptions for the labeling of antibodies with enzymes are described in Conjugation of enzymes to antibodies, Winston S. E., et al., 2001, Curr. Protoc. Mol. Biol., Chapter 11, Unit 11.1 and Antibodies—A Laboratory Manual, E. Harlow and D. Lane, Cold Spring Harbor Laboratory, 1988, Chapter 9, Labeling Antibodies, pp 342-358. This chapter includes coupling antibodies to horseradish peroxidase (pp 344-348), alkaline phosphatase (pp 349-350) and β-galactosidase (pp 350-353).

Conjugation of Antibodies to Specific Fluorochromes

Fluorescent reporters such as Cy3 or Cy5 can be used to generate specific fluorescently-tagged antibodies against specific GPI-linked proteins. These reagents will facilitate a cellular genotoxicity, multiplexed HCA and quantitative immunocytochemistry approach for use on optical imaging instrumentation (such as the GE Healthcare InCell 2000). This multiplex HCA approach could be correlated with other cytotoxic markers such as cell viability (using the CellTiter-Glo® Luminescent and CellTiter-Fluor Cell Viability Assays, Promega), micro-nuclei analysis (using the InCell 2000 Micronuclei Analysis Module, GE Healthcare), mitochondrial membrane integrity (using the HCS Mitochondrial Health Kit, Invitrogen).

Conjugation of antibodies to Cyanine dyes—The following cyanine antibody labeling kits are available from GE Healthcare Cy2, Cy3 and Cy5 Ab Labeling Kit (Cat. No. PA32000, PA33000 and PA35000 respectively). The dyes are provided in a reactive format as a bisfunctional NHS-ester for the labeling of antibodies containing primary amines (e.g. lysines). The dyes are water soluble, highly fluorescent and are useful as fluorescent labels for biological compounds. The Cy2 reagent produces an intense green signal (detected using fluorescein filters), Cy3 is an orange fluorescing cyanine (detected using rhodamine filters) and Cy5 produces an intense signal in the far-red region of the spectrum.

The antibody conjugating protocol described below has been designed for the preparation of Cy3-labeled antibodies but is equally applicable for labeling antibodies with either Cy2- or Cy5. It is designed to label 0.1 mg protein to a final molar dye:antibody ratio between 4 and 12. (http://www5.gelifesciences.com).

The antibody is dissolved at 1 mg/ml in 50 mM phosphate buffer pH 7.0 and 5 μl of the coupling buffer (1 M sodium carbonate buffer, pH 9) is added to 100 μl of the antibody solution. This solution is transferred to the reactive cyanine dye mixture and incubated at room temperature for 30 min.

To separate the cyanine dye conjugated antibody from un-reacted Cy3, G-25 Sephadex gel filtration is used. Elution buffer (3.0 ml) consisting of PBS pH 7.2, containing 0.1% NaN₃ is added to the gel filtration column. The antibody-labeling mixture is transferred to the column and 1.0 ml of elution buffer added. As the mixture enters the column, a pink band of labeled antibody separates from the un-conjugated dye. An additional 1.0 ml of elution buffer is added and the faster moving pink band is collected.

Conjugation of Antibodies to Fluorescein

Fluorescein is a small organic molecule, and is typically conjugated to antibodies via primary amines. Usually, between 3 and 6 molecules are conjugated to each antibody; higher conjugations can result in solubility problems as well as internal quenching. Thus an antibody will usually be conjugated in several parallel reactions to different amounts of dye, and the resulting reagents (after application to cells and antibody titration) will be compared for brightness in order to choose the optimal conjugation ratio. Fluorescein is excited at 488 nm and emits at 530 nm. (See http://www.drmr.com/abcon/FITC.html for details.)

Reactive fluorescein isothiocyanate (FITC) is available from Sigma. A range of FITC to antibody concentrations should be compared when conjugating an antibody (e.g. 10, 40, 80, 160, 320 μg FITC per mg antibody).

In order to remove traces of the preservative NaN₃ the antibody preparation should be subjected to overnight dialysis in 500 mM carbonate, pH 9.5. The covalent conjugation of the antibody to FITC is achieved by dissolving FITC in anhydrous DMSO. This solution is mixed with the antibody at a ratio of 40-80 μg FITC per mg of antibody. The reaction is wrapped in foil and incubated at room temperature for 1 hour. The un-reacted FITC is removed by dialysis in 10 mM Tris, 150 mM NaCl, 0.1% NaN₃, pH 8.2.

All the fluorescent dye-conjugated antibodies can be used in quantitative immuno-cytochemistry experiments when performed on imaging platforms such as the InCell 2000 GE Healthcare.

A comprehensive description for the labeling of antibodies with other fluorescent compounds and activated derivatives is described in Antibodies—A Laboratory Manual, Ed. Harlow and David Lane, Cold Spring Harbor Laboratory, 1988, Chapter 9, Labeling Antibodies, pp 353-358. This chapter includes coupling antibodies to i) the isothiocyanate derivatives of fluorescein and rhodamine and ii) the dichlorotriazinylamine derivatives of fluorescein. In addition the webpage http://www.drmr.com/abcon/ describes protocols for the conjugation of antibodies to fluorescent reagents such as Texas Red, phycoerythrin, allophycocyanin and cascade blue.

Enzymatic Assay Method

Cells under investigation will be exposed to a range of genotoxic compounds for example hydrogen peroxide, (10-100 μM), benzo(a)pyrene (30 μM), pyridol[4,3-b]indole (10 μM), phenylimidazol[4,5-b]pyridine (25-300 μM), nitrosodimethylamine (150-300 mM), aflatoxin (0.3-3 μM), 7,12-dimethyl-1,2-benz[a]anthracene (100 μM) or ethyl-N-nitrosurea (100 μM). All of these compounds are available from Sigma and are known to exhibit varying genotoxic effects (see Winter, H. K., et al., 2008, Mut. Res., 657, 133-139 and Bryce, S. M., et al., 2008, Environ. Mol. Mutagenesis, 49, 256-264).

Examples of control compounds include, methyl methane sulfonate (1-100 μg/ml) as a known strong genotoxic compound, 4-nitroquinolone-N-oxide (0.01-1 μg/ml) is a strong genotoxic and cytotoxic compound. Non-genotoxic compounds include the cytotoxic agent phenformin (1-250 μg/ml) and the non-toxic mannitol (10μg-2 mg/ml).

Adherent cells are grown in the presence of the appropriate cell culture medium in a 96- (20,000 pre well) or 384- (5,000 cells per well) well cell culture plate and incubated overnight at 37° C. in a 5% atmosphere of CO₂. An aliquot (e.g. 5 μl) of a suitable test compound (e.g. hydrogen peroxide, methyl methane sulfonate) dissolved or suspended in a non-toxic solvent is added to each well and the plate incubated for 1-48 hr at 37° C. in 5% CO₂. The medium supplemented with the genotoxic compound under investigation is changed every 24 hr. After the appropriate incubation time the medium is removed and the adherent cells are washed twice with phosphate buffered saline (PBS). The enzyme-conjugated antibody is diluted 1:250 to 1:10,000 in PBS and the appropriate volume added to the cells (96- and 384-well e.g. 50 and 15 μl respectively). The plates are incubated for >2 hr at room temperature to facilitate antibody antigen interaction. Residual unbound enzyme-conjugated antibody is removed by further washing with PBS.

For a β-galactosidase-conjugated antibody an appropriate luminescent substrate (e.g. 5-acetylaminofluorescein di-b-D-galactopyranoside or 5-acetylaminofluorescein di-b-D-galactopyranoside both from Invitrogen or 5-lodo-3-indolyl-beta -D-galactopyranoside from Sigma) is added to each well and the plate incubated for 1 to 18 hour (s) at 37° C. in 5% CO₂. Alternatively the Promega Beta-Glo Assay System could be used.

For luciferase-conjugated antibodies appropriate luminescent reagents are used e.g. ONE-GIo™ Luciferase Assay Reagent from Promega. The Dual-GIo™ Luciferase Assay reagent could also be used to monitor both the expression of CD55 and CD59 in the same cell line. In this instance the respective antibodies are conjugated to either Firefly or Renilla luciferase respectively. The dual luciferase assay is a fast two step reaction, i) the Firefly substrate (Beetle luciferin) is added and the response monitored ii) The “Stop and Glo buffer” is added this contains a Firefly luciferase quencher and the Renilla luciferase substrate colenterazine. These reagents are added to each well and the plate incubated for 1 to 18 hour (s) at 37° C. in a 5% CO₂ atmosphere.

A change in the optical signal (e.g. fluorescence or luminescence) is read using a plate reader, photomultiplier device or imager (e.g. Leadseeker, GE Healthcare). The value is compared to that generated in the presence of control reagents i.e. known genotoxic and non-genotoxic compounds.

Enzymatic Assays Involving the Addition of Phospholipase C

Phospholipase C (PLC) cleaves the phospho-glycerol bond linking GPI-moeity to the anchored protein. Thereby PLC releases the attached protein from the membrane associated GPI anchor. PLC therefore can be used to liberate these proteins into the cell culture medium. Mutations at the PigA locus will reduce the presence of these proteins at the cell surface. Once liberated the presence of the protein in the cell medium can be monitored enzymatically by either using enzyme-conjugated antibodies or directly providing the released protein is enzymatically active.

Adherent or suspension cells are grown, exposed to i) the genotoxic compound and ii) the appropriate enzyme-conjugated antibody. Those cells expressing GPI-linked enzymes such as acetylcholinesterase are simply exposed to PLC after henotoxic insult.

After steps i) and ii) have been completed the cells are washed twice in PBS and then exposed to PLC (96- and 384-well 50 and 15 μl respectively at 1.0 μg/ml). The cell culture plates are incubated for 1 hr at 37° C. After which an aliquot (e.g. 10 μl), of the supernatant is removed and used directly in an enzyme assay in combination with the appropriate substrates. For example fluorescent or luminescent luciferase or β-galactosidase substrates are used for the enzyme-conjugated antibody format while direct assays are performed for the released acetylcholinesterase and alkaline phosphatase activities.

Acetylcholinesterase and alkaline phosphatase activities can be measured using commercially available kits and substrates. For example, Cell technology Inc., supply the aCella Acetylcholinesterase assay. This involves using the substrate, acetylcholine in combination with a series of coupled enzyme reactions. First, acetylcholine is hydrolyzed by acetylcholinesterase to yield acetate and choline. These products are then utilised in a coupled enzyme reaction that results in consumption of ATP. Finally the residual ATP concentration is measured by the luciferase/luciferin method.

Alkaline phosphatase activity can be measured using several commercially available kits including the SensoLyte Luminescent Alkaline Phosphatase Assay Kit (Anaspec), the Luminometric Alkaline Phosphatase Assay Kit (Amplite). These kits provide sensitive chemiluminescent substrates to quantify alkaline phosphatase activity in solutions, in cell extracts and in live cells. Vector Laboratories market the Vector Red Alkaline Phosphatase Substrate Kit. The Vector Red substrate can be used for either determining enzymatic activity or for cell staining during HCA optical imaging. Enzo Diagnostics market the NBT/BCIP Alkaline Phosphatase Substrate Kit for the visualization of phosphatase reactions using nitro-blue tetrazolium chromogen and bromochloroindolyl phosphate substrate. The dephosphorylation of BCIP by alkaline phosphatase mediates its reaction with NBT to form a bluish-purple precipitate.

Quantitative Multiplex High Content Screening

Cells under investigation will be exposed to a range of genotoxic compounds for example hydrogen peroxide, (10 -100 μM), benzo(a)pyrene (30 μM), pyridol[4,3-b]indole (10 μM), phenylimidazol[4,5-b]pyridine (25-300 μM), nitrosodimethylamine (150-300 mM), aflatoxin (0.3-3 μM), 7,12-dimethyl-1,2-benz[a]anthracene (100 μM) or ethyl-N-nitrosurea (100 μM). All of these compounds are available from Sigma and are known to exhibit varying genotoxic effects (see Winter, H. K., et al., 2008, Mut. Res., 657, 133-139 and Bryce, S. M., et al., 2008, Environ. Mol. Mutagenesis, 49, 256-264).

Examples of control compounds include, methyl methane sulfonate (1-100 μg/ml) as a known strong genotoxic compound, 4-nitroquinolone-N-oxide (0.01-1 μg/ml) is a strong genotoxic and cytotoxic compound. Non-genotoxic compounds include the cytotoxic phenformin (1-250 μg/ml) and the non-toxic mannitol (10 μg-2 mg/ml).

Adherent cells are grown in the presence of the appropriate cell culture medium in a 96- (20,000 pre well) or 384- (5,000 cells per well) well cell culture plate and incubated overnight at 37° C. in 5% CO₂. An aliquot (e.g. 5 μl) of a suitable test compound (e.g. hydrogen peroxide, methyl methane sulfonate) dissolved or suspended in a non-toxic solvent is added to each well and the plate incubated for 1-48 hr at 37° C. in 5% CO₂. The medium supplemented with the genotoxic compound under investigation is changed every 24 hr. After the appropriate incubation time the medium is removed and the adherent cells are washed twice with phosphate buffered saline (PBS). Prior to the addition of the antibody the cells are subjected to a formalin-mediate cell fixation protocol.

This involves washing the cells with PBS and then adding PBS supplemented with 5% formalin and 2.5 μM of the nuclear stains Hoechst or propidium iodide (Molecular Probes) for 30 min at room temperature. After the formalin solution is decanted and the cells are washed twice with PBS. The fluorescently-conjugated antibody (or antibodies) is diluted 1:250 to 1:10,000 in PBS and the appropriate volume added to the cells (96- and 384-well e.g. 50 and 15 μl respectively). The plates are incubated for >2 hr at room temperature to facilitate antibody antigen interaction. Residual unbound enzyme-conjugated antibody is removed by further washing with PBS, the final wash being retained. Quantitative immuno-cytochemistry is then performed using imagers such as the GE Healthcare InCell 2000. Excitation laser are set at the appropriate wavelength for visualisation of the respective fluorescent makers.

In addition the method could involve staining the cells with fluorescent Calcein AM (BD Biosciences). This dye is a cell viability indicator. It is a non-fluorescent, cell permeable compound that is hydrolyzed by intracellular esterases into a fluorescent anion. It is used to fluorescently label viable cells. Calcein exhibits absorption and emission spectra corresponding to 494 and 517 nm respectively. The cells are labeled in situ using 1 μM Calcein AM in HBSS supplemented with 0.1% BSA for 60 minutes at 37° C. The dye is removed from the cells by PBS washes. Subsequent manipulations are then performed e.g. when used in combination with propidium iodide as live-dead cellular reporters. This involves measuring the decrease in propidium iodide staining and the increase in Calcein staining, following induction of mutations (Chen. et al., 2001, Cancer Research, 61, 654-658).

This multiplex quantitative HCA approach could be correlated with other cytotoxic markers such as cell viability (using the CellTiter-Glo® Luminescent and CellTiter-Fluor Cell Viability Assays, Promega), micro-nuclei analysis (using the InCell 2000 Micronuclei Analysis Module, GE Healthcare), mitochondrial membrane integrity (using the HCS Mitochondrial Health Kit, Invitrogen).

Suspension Cells

For assays using suspension cells similar protocols to those described above are followed (e.g. cell numbers, reagent exposure times, etc.) however the major difference is that on changing the medium, PBS washes or exposure to antibodies the cells are first pelleted by centrifuging the cell culture plate at 1,000 rpm for 5 min in an Eppendorf plate Centrifuge 5403.

1. An in vitro method of measuring the genotoxicity of an agent in human cells comprising the steps of:
(i) providing an in vitro culture of human cells comprising the phosphatidylinositol glycan biosynthesis class A (PigA) gene and expressing at least one glycosylphosphatidylinositol (GPI) anchor;
(ii) determining a control level of said GPI-anchor or a GPI-anchored protein in said cells;
(iii) exposing the cells to an agent;
(iv) determining a treatment level of the GPI-anchor or said GPI-anchored protein;
(v) comparing said treatment level with said control level of the GPI-anchor or the GPI-anchored protein,
wherein a difference in the levels of the GPI-anchor or the GPI-anchored protein is indicative of the genotoxicity of said agent.

2. The method of claim 1, wherein the levels of the GPI anchor is determined using a GPI- anchor binding moiety.

3. The method of claim 2, wherein the said GPI-anchor binding moiety is selected from the group consisting of antibody, proaerolysin, aerolysin and alphatoxin.

4. The method of claim 1, wherein the GPI anchored protein is selected from the group consisting of CD14, CD16, CD24, CD28, CD48, CDw52, CD55, CD56, CD58, CD59, CD66a, CD66c, CD66d, CD66E, CD67, CD73, CD87, CD90, CD108, CD157, acetylcholinesterase (AchE), alkaline phosphatase (NAP), Urokinase type plasminogen activating receptor (uPAR), JMH protein, GDNFR, CNTFR, TAG-1, PrP, Glypican protein, Semaphorin 7, CEA, GFR, Ly6G, transferrin receptor, Contactin (F3) and T-cadherin.

5. The method of claim 4, wherein the GPI anchored protein is selected from the group consisting of CD55, CD59, acetylcholinesterase (AchE) and alkaline phosphatase (NAP).

6. The method of claim 1, wherein the cells are selected from the group consisting of hepatocytes, cardiomyoctyes, blood cells, haematopoietic cells, stem cells, neuronal cells, renal cells, placental cells, osteoblasts, osteocytes, osteoclasts, spleen cells, pancreatic cells dermal cells and cells isolated from the gut.

7. The method of claim 1, wherein the cells are selected from a group of cell lines consisting of human monocytic leukaemia cell line, hepatoma cell line Hep3B, hepatoma cell line HepG2, erythroleukemia tumor cell line K562, CHO, Hela and NTera-2 human pluripotent embryonal carcinoma cell line.

8. The method of claim 1, wherein the levels of GPI anchored protein are determined by quantitative immunocytochemistry.

9. The method of claim 1, wherein the levels of GPI anchored protein are determined by Surface Plasmon Resonance (SPR).

10. The method of claim 1, wherein the levels of GPI anchored protein are determined by measurement of GPI anchored protein enzymatic activity.

11. The method of claim 10, wherein said enzymatic activity is acetylcholinesterase or alkaline phosphatase activity.

12. The method of claim 1, wherein the GPI anchored protein is released from the cells prior to determining the level of GPI anchored protein.

13. The method of claim 12, wherein the GPI anchored protein is released from the cells by treatment with phospholipase C.

14. The method of claim 1, wherein said method is a multiplex method which additionally comprises measuring a cellular event which is unrelated to the levels of GPI anchor or GPI anchored protein.

15. The method of claim 14, wherein said cellular event is selected from the group consisting of cell viability, micro-nuclei presence, mitochondrial membrane integrity, cell proliferation and apoptosis.

16-20. (canceled)
